SEQUENCE LISTING (38 sequences)

SEQ ID NO: 1 - protein, 5 aa, Artificial Sequence
  VH-CDR1 of mouse, chimeric and humanized 70E11 antibodies
SYNVH

SEQ ID NO: 2 - protein, 17 aa, Artificial Sequence
  VH-CDR2 of mouse, chimeric and humanized 70E11 antibodies
TIYPGNLATSYNQKFKG

SEQ ID NO: 3 - protein, 6 aa, Artificial Sequence
  VH-CDR3 of mouse, chimeric and humanized 70E11 antibodies
SGTMDY

SEQ ID NO: 4 - protein, 12 aa, Artificial Sequence
  VL-CDR1 of mouse, chimeric and humanized 70E11 antibodies
RASSSISSTYLH

SEQ ID NO: 5 - protein, 7 aa, Artificial Sequence
  VL-CDR2 of mouse, chimeric and humanized 70E11 antibodies
NTQNLAS

SEQ ID NO: 6 - protein, 10 aa, Artificial Sequence
  VL-CDR3 of mouse, chimeric and humanized 70E11 antibodies
QQFGGYPLIT

SEQ ID NO: 7 - protein, 5 aa, Artificial Sequence
  VH-CDR1 of mouse, chimeric and humanized 149G11 antibodies
NYWIH

SEQ ID NO: 8 - protein, 17 aa, Artificial Sequence
  VH-CDR2 of mouse, chimeric and humanized 149G11 antibodies
DIYPGGRYSNYNEKFRG

SEQ ID NO: 9 - protein, 8 aa, Artificial Sequence
  VH-CDR3 of mouse, chimeric and humanized 149G11 antibodies
YYESAMDF

SEQ ID NO: 10 - protein, 17 aa, Artificial Sequence
  VL-CDR1 of mouse, chimeric and humanized 149G11 antibodies
KSSQNLLYNSNQKSYLA

SEQ ID NO: 11 - protein, 7 aa, Artificial Sequence
  VL-CDR2 of mouse, chimeric and humanized 149G11 antibodies
WASTRES

SEQ ID NO: 12 - protein, 9 aa, Artificial Sequence
  VL-CDR3 of mouse, chimeric and humanized 149G11 antibodies
QQYYNYPFT

SEQ ID NO: 13 - protein, 115 aa, Artificial Sequence
  VH of mouse and chimeric 70E11 antibodies
QVQLQQPGTDLVKPGASVRMSCKASGYTFTSYNVHWVKQTPGQGLEWIGTIYPGNLATSYNQKFKGKATLTADKSSTTAYMQLSSLTSEDSAVYYCARSGTMDYWGQGTTLTVSS

SEQ ID NO: 14 - protein, 115 aa, Artificial Sequence
  VH of 70E11-VH1VL1
QVQLVQSGAEVKKPGASVKVSCKASGYTFTSYNVHWVRQAPGQGLEWMGTIYPGNLATSYNQKFKGRVTMTRDTSTSTVYMELSSLRSEDTAVYYCARSGTMDYWGQGTTVTVSS

SEQ ID NO: 15 - protein, 115 aa, Artificial Sequence
  VH of 70E11-VH2VL2, 70E11-VH2VL3 and 70E11- VH2VL4
QVQLVQSGAEVKKPGASVKVSCKASGYTFTSYNVHWVRQAPGQGLEWMGTIYPGNLATSYNQKFKGRVTLTADTSTSTVYMELSSLRSEDTAVYYCARSGTMDYWGQGTTVTVSS

SEQ ID NO: 16 - protein, 115 aa, Artificial Sequence
  VH of 70E11-VH3VL2, 70E11-VH3VL3 and 70E11- VH3VL4
QVQLVQSGAEVKKPGASVKVSCKASGYTFTSYNVHWVRQAPGQGLEWIGTIYPGNLATSYNQKFKGRVTLTADKSTSTVYMELSSLRSEDTAVYYCARSGTMDYWGQGTTVTVSS

SEQ ID NO: 17 - protein, 115 aa, Artificial Sequence
  VH of 70E11-VH4VL2, 70E11-VH4VL3 and 70E11- VH4VL4
QVQLVQSGAEVKKPGASVKVSCKASGYTFTSYNVHWVKQTPGQGLEWIGTIYPGNLATSYNQKFKGRVTLTADKSTSTVYMELSSLRSEDTAVYYCARSGTMDYWGQGTTVTVSS

SEQ ID NO: 18 - protein, 115 aa, Artificial Sequence
  VH of 70E11-VH5VL2, 70E11-VH5VL3 and 70E11- VH5VL4
QVQLVQSGAEVKKPGASVKVSCKASGYTFTSYNVHWVKQTPGQGLEWIGTIYPGNLATSYNQKFKGKATLTADKSTSTVYMELSSLRSEDTAVYYCARSGTMDYWGQGTTVTVSS

SEQ ID NO: 19 - protein, 117 aa, Artificial Sequence
  VH of mouse and chimeric 149G11 antibodies
QVQLQQSGTELVRPGTSVKMSCRTAGYTFTNYWIHWVKQRPGHGLEWIGDIYPGGRYSNYNEKFRGKATLTADTSSSTAYMELSSLTSEDSAIYYCARYYESAMDFWGQGTSVTVSS

SEQ ID NO: 20 - protein, 117 aa, Artificial Sequence
  VH of 149G11-VH1VL1
QVQLVQSGAEVKKPGASVKVSCKASGYTFTNYWIHWVRQAPGQGLEWMGDIYPGGRYSNYNEKFRGRVTMTRDTSTSTVYMELSSLRSEDTAVYYCARYYESAMDFWGQGTTVTVSS

SEQ ID NO: 21 - protein, 117 aa, Artificial Sequence
  VH of 149G11-VH2VL2 and 149G11-VH2VL3
QVQLVQSGAEVKKPGASVKVSCKASGYTFTNYWIHWVRQAPGQGLEWIGDIYPGGRYSNYNEKFRGRVTLTADTSTSTVYMELSSLTSEDTAVYYCARYYESAMDFWGQGTTVTVSS

SEQ ID NO: 22 - protein, 117 aa, Artificial Sequence
  VH of 149G11-VH3VL2 and 149G11-VH3VL3
QVQLVQSGAEVKKPGASVKVSCKASGYTFTNYWIHWVKQAPGQGLEWIGDIYPGGRYSNYNEKFRGRVTLTADTSTSTVYMELSSLTSEDTAVYYCARYYESAMDFWGQGTTVTVSS

SEQ ID NO: 23 - protein, 117 aa, Artificial Sequence
  VH of 149G11-VH4VL2 and 149G11-VH4VL3
QVQLVQSGAEVKKPGASVKVSCKASGYTFTNYWIHWVKQRPGHGLEWIGDIYPGGRYSNYNEKFRGRVTLTADTSTSTVYMELSSLTSEDTAVYYCARYYESAMDFWGQGTTVTVSS

SEQ ID NO: 24 - protein, 109 aa, Artificial Sequence
  VL of mouse and chimeric 70E11 antibodies
ENVVTQSPAIMSASPGEKVTMTCRASSSISSTYLHWYQQKSGASPKLWIYNTQNLASGVPARISGSGSGTSYSLTISSVEAEDAATYYCQQFGGYPLITFGAGTKLELK

SEQ ID NO: 25 - protein, 109 aa, Artificial Sequence
  VL of 70E11-VH1VL1
EIVLTQSPGTLSLSPGERATLSCRASSSISSTYLHWYQQKPGQAPRLLIYNTQNLASGIPDRFSGSGSGTDFTLTISRLEPEDFAVYYCQQFGGYPLITFGAGTKLELK

SEQ ID NO: 26 - protein, 109 aa, Artificial Sequence
  VL of 70E11-VH2VL2, 70E11-VH3VL2, 70E11-VH4VL2 and 70E11-VH5VL2
EIVLTQSPGTLSLSPGERATLSCRASSSISSTYLHWYQQKPGQAPKLLIYNTQNLASGIPDRFSGSGSGTSFTLTISRLEPEDFAVYYCQQFGGYPLITFGAGTKLELK

SEQ ID NO: 27 - protein, 109 aa, Artificial Sequence
  VL of 70E11-VH2VL3, 70E11-VH3VL3, 70E11-VH4VL3 and 70E11-VH5VL3
EIVLTQSPGTLSLSPGERATLSCRASSSISSTYLHWYQQKPGASPKLLIYNTQNLASGIPDRFSGSGSGTSYTLTISRLEPEDFAVYYCQQFGGYPLITFGAGTKLELK

SEQ ID NO: 28 - protein, 109 aa, Artificial Sequence
  VL of 70E11-VH2VL4, 70E11-VH3VL4, 70E11-VH4VL4 and 70E11-VH5VL4
EIVLTQSPGTLSLSPGERATMTCRASSSISSTYLHWYQQKPGASPKLLIYNTQNLASGVPARFSGSGSGTSYTLTISRLEPEDFAVYYCQQFGGYPLITFGAGTKLELK

SEQ ID NO: 29 - protein, 113 aa, Artificial Sequence
  VL of mouse and chimeric 149G11 antibodies
DIVMSQSPSSLAVSVGEKVTVNCKSSQNLLYNSNQKSYLAWFQQKPGQSPKLLIYWASTRESGVPDRFTGSGSGTDFTLTISSVKAEDLAVYYCQQYYNYPFTFGSGTKLEIK

SEQ ID NO: 30 - protein, 113 aa, Artificial Sequence
  VL of 149G11-VH1VL1
DIVMTQSPDSLAVSLGERATINCKSSQNLLYNSNQKSYLAWYQQKPGQPPKLLIYWASTRESGVPDRFSGSGSGTDFTLTISSLQAEDVAVYYCQQYYNYPFTFGAGTKLELK

SEQ ID NO: 31 - protein, 113 aa, Artificial Sequence
  VL of 149G11-VH2VL2, 149G11-VH3VL2 and 149G11- VH4VL2
DIVMTQSPDSLAVSLGERATINCKSSQNLLYNSNQKSYLAWFQQKPGQPPKLLIYWASTRESGVPDRFSGSGSGTDFTLTISSLQAEDLAVYYCQQYYNYPFTFGAGTKLELK

SEQ ID NO: 32 - protein, 113 aa, Artificial Sequence
  VL of 149G11-VH2VL3, 149G11-VH3VL3 and 149G11- VH4VL3
DIVMTQSPDSLAVSLGERATVNCKSSQNLLYNSNQKSYLAWFQQKPGQSPKLLIYWASTRESGVPDRFSGSGSGTDFTLTISSLQAEDLAVYYCQQYYNYPFTFGAGTKLELK

SEQ ID NO: 33 - protein, 330 aa, Artificial Sequence
  Human IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 107 aa, Artificial Sequence [sequence omitted]

SEQ ID NO: 35 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 36 - protein, 312 aa, Macaca fascicularis
MAFLVAPPMQFVYLLKTLCVFNMVFAKPGFSETVFSHRLSFTVLSAVGYFRWQKRPHLLPVSPLGRSMRWCLFLIWAQGLRQAPLASGMMTGTIETTGNISAKKGGSVILQCHLSSTMAQVTQVNWEQHDHSLLAIRNAELGWHIYPAFKDRVAPGPGLGLTLQSLTMNDTGEYFCTYHTYPDGTYRGRIFLEVLESSVAEHSARFQIPLLGAMAMMLVVICIAVIVVVVLARKKKSLRIHSVESGLQRKSTGQEEQIPSAPSPPGSCVQAEAAPAGLCGEQQGDDCAELHDYFNVLSYRSLGSCSFFTETG

SEQ ID NO: 37 - protein, 241 aa, Mus musculus
MHGWLLLVWVQGLIQAAFLATGATAGTIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSVAQFQTAPLGGTMAAVLGLICLMVTGVTVLARKKSIRMHSIESGLGRTEAEPQEWNLRSLSSPGSPVQTQTAPAGPCGEQAEDDYADPQEYFNVLSYRSLESFIAVSKTG

SEQ ID NO: 38 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

(19) United States
(12) Patent Application Publication
(10) Pub. No.: US 2022/0396617 A1
(43) Pub. Date: Dec. 15, 2022

(54) ANTIBODIES BINDING TIGIT AND USES THEREOF

(71) Applicant: Beijing Mabworks Biotech Co. Ltd., Beijing (CN)

(72) Inventors: Jiangmei LI, Beijing (CN); Wenqi HU, Beijing (CN); Feng LI, Beijing (CN)

(21) Appl. No.: 17/460,530

(22) Filed: Aug. 30, 2021

(30) Foreign Application Priority Data
Jun. 10, 2021 (CN) 202110650525.X

(51) Int. Cl. C07K 16/28; A61P 35/00

(52) U.S. Cl. CPC C07K 16/2803; A61P 35/00; C07K 2317/732; C07K 2317/24; C07K 2317/92; A61K 2039/505

(57) ABSTRACT

An isolated monoclonal antibody, or an antigen-binding portion thereof, that specifically binds human TIGIT. A nucleic acid molecule encoding the antibody or antigen-binding portion thereof, an expression vector, a host cell and a method for expressing the antibody or antigen-binding portion thereof are also provided. The present disclosure further provides an immunoconjugate, a bispecific molecule, a chimeric antigen receptor, an oncolytic virus and a pharmaceutical composition comprising the antibody or antigen-binding portion thereof, as well as a treatment method using the antibody or antigen-binding portion thereof.

### RELATED APPLICATIONS AND INCORPORATION BY REFERENCE

This application claims priority to Chinese Patent Application No. CN202110650525.X filed on Jun. 10, 2021.

The foregoing application, and all documents cited therein or during its prosecution (“appln cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

Citation or identification of any document in this application is not an admission that such document is available as prior art to the present disclosure.

### SEQUENCE STATEMENT

The instant application contains a Sequence Listing, which has been submitted electronically and is hereby incorporated by reference in its entirety. Said ASCII copy, is named 55556_00064SL.txt and is 40 kb in size.

### FIELD OF THE INVENTION

The disclosure relates to an antibody or an antigen binding portion thereof specifically binding to human TIGIT, the preparation and use thereof, especially its use in treatment of diseases associated with TIGIT signaling, such as cancers and infective diseases.

### BACKGROUND OF THE INVENTION

The immune system provides protection against cancers. Dendritic cells take up and present cancer cell antigens released during oncogenesis to T cells, leading to T cell priming and activation, and then the activated T cells traffic to and kill cancer cells. Malignant cells, however, have evolved various ways to evade immune surveillance, by e.g., utilizing the inhibitory immune checkpoint pathways. These pathways consist of receptor-ligand pairs which, following receptor-ligand interaction, suppress the effector functions of T cells and natural killer (NK) cells and thereby impair anti-tumor immunity. For example, the tumor cells may highly express PD-L1 and PD-L2, which bind to PD-1 on T cell surfaces to induce T cell apoptosis, and also express CD47s, which bind to signal regulatory protein alpha (SIRPα) on the surfaces of macrophages to inhibit phagocytosis of tumor cells. The tumor-infiltrating lymphocytes face such immunosuppressive pressures and eventually become exhausted Immunotherapies targeting PD-1 and CLTA-4 have shown clinical benefits, and researches are expanding to more inhibitory immune checkpoints, including TIM-3, LAG-3 and TIGIT. Many of these molecules are found to be non-redundant, blockade of more than one may generate better anti-tumor immunity (Le Mercier I et al., (2015) Front Immunol. 6:418).

TIGIT, also referred to as T cell immunoglobulin and ITIM domain, Washington University cell adhesion molecule (WUCAM), V-set and transmembrane domain-containing protein 3 (Vstm3) and V-set and immunoglobulin domain-containing protein 9 (VSIG9), belongs to a constantly expanding family of PVR-like proteins. It consists of an extracellular immunoglobulin variable domain, a type I transmembrane domain and a short intracellular domain with one immunoreceptor tyrosine-based inhibitory motif (ITIM) and one immunoglobulin tyrosine tail (ITT)-like motif. The immunoglobulin variable domain shares sequence homology with other members of the PVR-like family, including CD226 (DNAM-1), CD96, CD155, CD111, CD112, CD113 and PVRL4.

TIGIT is expressed on activated CD8⁺ T and CD4⁺ T cells, natural killer (NK) cells, regulatory T cells, and follicular T helper cells in humans. It competes with CD226, a co-stimulatory receptor expressed on naïve and resting T cells, over CD155 binding, to counterbalance the costimulatory function of CD226, with its CD155 binding affinity much higher than CD226. The relative amount of TIGIT-CD155 binding versus CD226-CD155 binding determines whether a T cell undergoes activation or anergy. The TIGIT-CD155 interaction may block T cell receptor (TCR) signaling, and inhibit pro-inflammatory cytokine production by CD4⁺ T cells (Shibuya K et al., (1999) Immunity 11:615-623; Lozano E et al., (2013) J Immunol 191:3673-3680). TIGIT expression is also found in about 20-90% resting NK cells, which level is increased following acute or chronic virus infection or oncogenesis. The engagement of TIGIT with CD155 initiates major inhibitory signaling in human NK cells via the ITT-like motif, and decreases these cells' reactions to tumor cells and capabilities to release interferon-α (Holder K A, Grant M D. (2020) Front Cell Infect Microbiol. 10:175; Stanietsky N et al., (2009) Proc Natl Acad Sci USA 106:17858-17863; Liu S et al., (2013) Cell Death Differ 20:456-464). TIGIT further binds CD112 and PVRL3 with much weaker affinities.

Studies have shown TIGIT inhibits innate immunity and adaptive immunity through multiple ways. First, dendritic cells, when bound with TIGIT via cell surface CD155, acquire a tolerogenic phenotype, resulting in decreased IL-12 and increased IL-10 release, thereby suppressing antigen presenting molecule upregulation, T cell proliferation and cytokine secretion (Yu X et al., (2009) Nat. Immunol. 10: 48-57). Second, TIGIT has direct inhibitory effects on immune cells. For example, the TIGIT pathway may weaken TCR driven activation signals, inhibit T cell proliferation and effector function, while CD8⁺ tumor infiltrating lymphocytes that express TIGIT at a high level have reduced pro-inflammatory cytokine production and impaired degranulation ability (Kurtulus S et al., (2015) J. Clin. Invest. 125: 4053-4062). NK cells are the most important in early stages of cancer elimination. Their TIGIT expressions are negatively correlated with their abilities to secrete IFN-γ and kill CD155⁺ cells, and TIGIT⁺ NK cells are more likely to be suppressed by MDSC than TIGIT⁻ counterparts. TIGIT blockade may elicit NK cells' anti-tumor immunity (Wang F et al., (2015) Eur. J. Immunol. 45: 2886-2897; Manieri N A et al., (2017) Trends Immunol. 38(1): 20-28; Zhang Q et al., (2018) Nat. Immunol. 19: 723-732). Third, TIGIT is constitutively expressed in most Tregs, and TIGIT⁺ Tregs are more immunosuppressive than TIGIT⁻ Tregs in Th1 and Th17 response suppression and T cell function suppression (Joller N., et al., (2014) Immunity 40: 569-581; Kurtulus S et al., (2015) supra).

A significant body of work has highlighted the great therapeutic potential of targeting TIGIT with antagonistic mAbs in a wide range of malignancies. Etigilimab (OMP-313M32), an anti-TIGIT mAb developed by OncoMed Pharmaceuticals, was tested for its safety and pharmacokinetics in a Phase I, dose-escalation study (NCT031119428) as a single agent or in combination with nivolumab (anti-PD-1 mAb) in treatment of various advanced or metastatic solid malignancies, including colorectal cancer, endometrial cancer, and pancreatic cancer. The Phase Ia trial showed etigilimab was well tolerated at doses up to 20 mg/kg. Another antagonistic antibody, Tiragolumab, developed by Roche, was found effective against solid cancers, especially non-small cell lung cancer, when used in combination with the PD-L1 inhibitor atezolizumab. More anti-TIGIT antibodies, including BMS-986207 (Bristol-Myers Squibb), BGB-A1217 (BeiGene), and AB154 (Arcus biosciences), are being tested in clinical trials as a single agent or in combination with other anti-tumor agents for treating solid tumors such as multiple myeloma and melanoma (Chauvin J, Zarour H M., (2020) Journal for ImmunoTherapy of Cancer 8:e000957). Studies further showed that the constant regions of the anti-TIGIT antibodies may bind FcγRs on and activate myeloid cells, leading to enhanced antigen presentation, and cytokine and chemokine production, thereby promoting robust perforin and granzyme B release by T cells (Han J H et al., (2020) Front Immunol. 11:573405).

During chronic viral infections, effector cell functions are severely impaired. When the TIGIT signaling is blocked by an anti-TIGIT antibody, CD8⁺ T cells may restore their anti-viral activities. It was reported that the increase of TIGIT expression on NK cells was associated with HIV-1 progression, and whether TIGIT blockade can rescue NK cells' anti-viral activities remains to be further elucidated (Holder K A, Grant M D. (2020) supra; Yin X et al., (2018) Front. Immunol. 9:2341).

There remains a need for more anti-TIGIT antibodies with improved pharmaceutical characteristics as they are important in regulating immune system functions.

### SUMMARY OF THE INVENTION

The present disclosure provides an isolated monoclonal antibody, for example, a mouse, human, chimeric or humanized monoclonal antibody, that binds to TIGIT (e.g., the human TIGIT, and monkey TIGIT), or an antigen-binding portion thereof. It has comparable or higher binding activity to human and monkey TIGIT proteins, comparable blocking activity on TIGIT-PVR interaction, comparable or better T cell activation activity, comparable or higher activity of inducing antibody dependent cell mediated cytotoxicity (ADCC) to TIGIT⁺ cells, and comparable or better anti-tumor effect, as compared to prior art antibodies such as Tiragolumab and Etigilimab.

The antibody or antigen-binding portion thereof of the disclosure can be used for a variety of applications, including treatment of TIGIT associated diseases.

Accordingly, in one aspect, the disclosure pertains to an isolated monoclonal antibody (e.g., a humanized antibody), or an antigen-binding portion thereof, that binds TIGIT, that may comprise i) a heavy chain variable region that may comprise a VH-CDR1 region, a VH-CDR2 region and a VH-CDR3 region, wherein the VH-CDR1 region, the VH-CDR2 region and the VH-CDR3 region may comprise amino acid sequences having at least 80%, 81%, 82%, 83%, 84%, 85%, 86%, 87%, 88%, 89%, 90%, 91%, 92%, 93%, 94%, 95%, 96%, 97%, 98%, or 99% identity to, or set forth in (1) SEQ ID NOs: 1, 2 and 3, respectively; or (2) SEQ ID NOs: 7, 8 and 9, respectively; and/or ii) a light chain variable region that may comprise a VL-CDR1 region, a VL-CDR2 region and a VL-CDR3 region, wherein the VL-CDR1 region, the VL-CDR2 region and the VL-CDR3 region may comprise amino acid sequences having at least 80%, 81%, 82%, 83%, 84%, 85%, 86%, 87%, 88%, 89%, 90%, 91%, 92%, 93%, 94%, 95%, 96%, 97%, 98%, or 99% identity to, or set forth in (1) SEQ ID NOs: 4, 5 and 6, respectively; or (2) SEQ ID NOs: 10, 11 and 12, respectively.

The isolated monoclonal antibody or the antigen-binding portion thereof of the disclosure may comprise a heavy chain variable region and a light chain variable region, wherein the VH-CDR1 region, VH-CDR2 region, VH-CDR3 region, VL-CDR1 region, VL-CDR2 region and VL-CDR3 region may comprise amino acid sequences having at least 80%, 81%, 82%, 83%, 84%, 85%, 86%, 87%, 88%, 89%, 90%, 91%, 92%, 93%, 94%, 95%, 96%, 97%, 98%, or 99% identity to, or set forth in (1) SEQ ID NOs: 1, 2, 3, 4, 5 and 6, respectively; or (2) SEQ ID NOs: 7, 8, 9, 10, 11 and 12, respectively.

The heavy chain variable region of the disclosure may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to, or set forth in any one of SEQ ID NOs: 13-23.

The light chain variable region of the disclosure may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to, or set forth in any one of SEQ ID NOs: 24-32.

The isolated monoclonal antibody or antigen-binding portion thereof of the disclosure may comprise a heavy chain variable region and a light chain variable region which may respectively comprise amino acid sequences having at least 80%, 81%, 82%, 83%, 84%, 85%, 86%, 87%, 88%, 89%, 90%, 91%, 92%, 93%, 94% 95%, 96%, 97%, 98%, or 99% identity to, or set forth in (1) SEQ ID NOs: 13 and 24; (2) SEQ ID NOs: 14 and 25; (3) SEQ ID NOs: 15 and 26; (4) SEQ ID NOs: 15 and 27; (5) SEQ ID NOs: 15 and 28; (6) SEQ ID NOs: 16 and 26; (7) SEQ ID NOs: 16 and 27; (8) SEQ ID NOs: 16 and 28; (9) SEQ ID NOs: 17 and 26; (10) SEQ ID NOs: 17 and 27; (11) SEQ ID NOs: 17 and 28; (12) SEQ ID NOs: 18 and 26; (13) SEQ ID NOs: 18 and 27; (14) SEQ ID NOs: 18 and 28; (15) SEQ ID NOs: 19 and 29; (16) SEQ ID NOs: 20 and 30; (17) SEQ ID NOs: 21 and 31; (18) SEQ ID NOs: 21 and 32; (19) SEQ ID NOs: 22 [symbol] 31; (20) SEQ ID NOs: 22 and 32; (21) SEQ ID NOs: 23 and 31; or (22) SEQ ID NOs: 23 and 32.

The isolated monoclonal antibody of the disclosure may comprise a heavy chain constant region and/or a light chain constant region. The heavy chain constant region may be an IgG1, IgG2, IgG3 or IgG4 heavy chain constant region, or a functional fragment thereof, preferably with FcγR binding affinity, such as human IgG1 heavy chain constant region having the amino acid sequence of SEQ ID NO: 33 or a functional fragment thereof. The light chain constant region may be kappa light chain constant region or a functional fragment thereof, such as human kappa light chain constant region having the amino acid sequence of SEQ ID NO: 34 or a functional fragment thereof. The N terminus of the heavy chain constant region is linked to the C terminus of the heavy chain variable region, and the N terminus of the light chain constant region is linked to the C terminus of the light chain variable region.

In certain embodiments, the antibody of the disclosure may comprise or consists of two heavy chains and two light chains connected by disulfide bonds, wherein each heavy chain comprises the heavy chain constant region, heavy chain variable region or CDR sequences mentioned above, and each light chain comprises the light chain constant region, light chain variable region or CDR sequences mentioned above. The antibody of the disclosure may be a full-length antibody, for example, of an IgG4, IgG1 or IgG2 isotype. The antibody or antigen binding portion thereof of the present disclosure in other embodiments may be a single chain antibody, or consists of antibody fragments, such as Fab or F(ab′)2 fragments.

The exemplary antibody or antigen binding portion thereof of the disclosure is antagonistic, which binds to human/monkey TIGIT to block TIGIT-PVR interaction, inducing T cell activation and ADCC against TIGIT positive cells, thereby providing in vivo anti-tumor effects.

The disclosure also provides an immunoconjugate comprising the antibody or the antigen binding portion thereof, linked to a therapeutic agent such as a cytotoxin or an anti-cancer agent. The disclosure also provides a bispecific molecule comprising the antibody or the antigen-binding portion thereof of the disclosure, linked to a second functional moiety (e.g., a second antibody) having a different binding specificity than the antibody or the antigen-binding portion thereof of the disclosure. In another aspect, the antibody or the antigen-binding portion thereof of the present disclosure can be made into part of a chimeric antigen receptor (CAR) or a T cell receptor (TCR). The disclosure further provides an immune cell with the CAR or TCR of the disclosure, such as a T cell and a NK cell. The antibody or the antigen binding portion thereof of the disclosure can also be encoded by or used in conjunction with an oncolytic virus.

The disclosure further provides a nucleic acid molecule encoding the antibody or antigen-binding portion thereof of the disclosure, as well as an expression vector comprising such a nucleic acid molecule and a host cell comprising such an expression vector. A method for preparing the anti-TIGIT antibody or antigen binding portion thereof using the host cell of the disclosure is provided, comprising steps of (i) expressing the antibody or antigen binding portion thereof in the host cell, and (ii) isolating the antibody or antigen binding portion thereof from the host cell or its cell culture.

The disclosure provides a composition comprising the antibody or antigen binding portion thereof, the immunoconjugate, the bispecific molecule, the immune cell, the oncolytic virus, the nucleic acid molecule, the expression vector, or the host cell of the disclosure, and a pharmaceutically acceptable carrier.

In another aspect, the disclosure provides a method for enhancing an immune response in a subject in need thereof, comprising administering to the subject a therapeutically effective amount of the composition of the disclosure. In some embodiments, the method comprises inducing T cell activation.

In another aspect, the disclosure provides a method for treating or alleviating cancer in a subject in need thereof, comprising administering to the subject a therapeutically effective amount of the composition of the disclosure. The cancer may be a solid cancer, including, but not limited to, liver cancer, rectal cancer, endometrial cancer, pancreas cancer, non-small cell lung cancer, multiple myeloma, and melanoma. In some embodiments, at least one additional anti-cancer antibody may be administered with the composition of the disclosure, such as an anti-PD-1 antibody, an anti-PD-L1 antibody, an anti-CTLA-4 antibody, and/or an anti-STAT3 antibody, especially an anti-PD-L1 antibody. In certain embodiments, the composition of the disclosure may be administered with a cytokine (e.g., IL-2 and/or IL-21), or a costimulatory antibody (e.g., an anti-CD137 and/or anti-GITR antibody). In another embodiment, the composition of the disclosure may be administered with a chemotherapeutic agent, which may be a cytotoxic agent.

In another aspect, the disclosure provides a method for treating or alleviating an infectious disease in a subject in need thereof, comprising administering to the subject a therapeutically effective amount of the antibody or antigen binding portion thereof. The infectious disease may be a chronic viral, bacterial, fungal or mycoplasma infection, such as a chronic HIV infection. In certain embodiments, at least one additional anti-infective agent may be administered with the antibody or antigen binding portion thereof of the disclosure, such as an anti-viral agent, an anti-bacterial agent, an anti-fungal agent, or an anti-mycoplasma agent.

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 shows increased IFN-γ release by T cells treated with anti-TIGIT antibodies at 100 μg/ml (A) or at different concentrations (B).

FIG. 2 shows the binding activities of the chimeric anti-TIGIT antibodies to HEK293A/human TIGIT cells (A), HEK293A/monkey TIGIT cells (B) and HEK293A/mouse TIGIT cells (C).

FIG. 3 shows the antibody dependent cell mediated cytotoxicity (ADCC) against HEK293A/human TIGIT cells induced by the chimeric 70E11 and 149G11 antibodies.

FIG. 4 shows the activities of the chimeric 70E11 and 149G11 antibodies to block TIGIT-PVR interaction.

FIG. 5 shows the binding capabilities of humanized 70E11 antibodies to HEK293A/human TIGIT cells (A), HEK293A/monkey TIGIT cells (B) and HEK293A/mouse TIGIT cells (C).

FIG. 6 shows the binding capabilities of humanized 149G11 antibodies to HEK293A/human TIGIT cells (A), HEK293A/monkey TIGIT cells (B) and HEK293A/mouse TIGIT cells (C).

FIG. 7 shows the activities of the humanized 70E11 antibodies (A) and humanized 149G11 antibodies (B) to block TIGIT-PVR interaction.

FIG. 8 shows IFN-γ secretion by T cells induced by the humanized anti-TIGIT antibodies in a dose-dependent manner.

FIG. 9 shows the ADCC against HEK293A/human TIGIT cells as induced by the humanized 70E11 antibodies (A) and humanized 149G11 antibodies (B).

FIG. 10 shows the ADCC against HEK293A/human TIGIT cells by PBMCs collected from Donor 1 (A), Donor 2 (B) and Donor 3 (C), as induced by the humanized anti-TIGIT antibodies.

FIG. 11 shows the average tumor size (A) and the average tumor weight (B) in C57 mice respectively treated with 149G11H2L3, 70E11H5L3 and Tiragolumab.

FIG. 12 shows the average tumor size in BALB/c mice treated with 149G11H2L3 alone or in combination with Tecentriq, 70E11H2L4 alone or in combination with Tecentriq, and Tiragolumab alone or in combination with Tecentriq (A), 149G11H2L3 alone or in combination with Tecentriq (B), 70E11H2L4 alone or in combination with Tecentriq (C), and Tiragolumab alone or in combination with Tecentriq (D).

### DETAILED DESCRIPTION OF THE INVENTION

To ensure that the present disclosure may be more readily understood, certain terms are first defined. Additional definitions are set forth throughout the detailed description.

The term “TIGIT” refers to T cell immunoglobulin and ITIM domain. The term “TIGIT” comprises variants, isoforms, homologs, orthologs and paralogs. For example, an antibody specific for a human TIGIT protein may, in certain cases, cross-react with a TIGIT protein from a species other than human, such as monkey. In other embodiments, an antibody specific for a human TIGIT protein may be completely specific for the human TIGIT protein and exhibit no cross-reactivity to other species or of other types, or may cross-react with TIGIT from certain other species but not all other species.

The term “human TIGIT” refers to a TIGIT protein having an amino acid sequence from a human, such as the amino acid sequence having a NCBI accession no. NP_776160.2 (Saleh R et al., (2020) Cancer Immunol Immunother 69(10): 1989-1999) or set forth in SEQ ID NO: 35. The term “monkey TIGIT” refers to a TIGIT protein having an amino acid sequence from a monkey, such as the amino acid sequence having a GenBank accession no. AFH31430.1 (Zimin A. V. et al., (2014) Biol. Direct 9(1): 20) or set forth in SEQ ID NO: 36. The term “mouse TIGIT” refers to a TIGIT protein having an amino acid sequence from a mouse, such as the amino acid sequence having a NCBI accession no. NP_001139797.1 (Schorer M et al., (2020) Nat Commun 11(1): 1288) or set forth in SEQ ID NO: 37.

The term “antibody” as referred to herein includes whole antibodies of e.g., IgG, IgA, IgD, IgE and IgM, and any antigen binding fragment (i.e., “antigen-binding portion”) or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1 q) of the classical complement system.

The term “antigen-binding portion” of an antibody (or simply “antibody portion”), as used herein, refers to one or more fragments of an antibody that retain the ability to specifically bind to an antigen (e.g., a TIGIT protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L} V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR); and (viii) a nanobody, a heavy chain variable region containing a single variable domain and two constant domains. Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds a TIGIT protein is substantially free of antibodies that specifically bind antigens other than TIGIT proteins). An isolated antibody that specifically binds a human TIGIT protein may, however, have cross-reactivity to other antigens, such as TIGIT proteins from other species. Moreover, an isolated antibody can be substantially free of other cellular material and/or chemicals.

The term “monoclonal antibody” as used herein refers to a population of substantially homogeneous antibodies, i.e., the individual antibodies comprising the population are identical except for possible naturally occurring mutations and/or post-translation modifications (e.g., isomerization, amidation) that may be present in minor amounts. Monoclonal antibodies are highly specific, being directed against a single antigenic site. In contrast to polyclonal antibody preparations which typically include different antibodies directed against different determinants (epitopes), the monoclonal antibodies are directed against a single determinant on the antigen.

The term “mouse antibody”, as used herein, is intended to include antibodies having variable regions in which both the framework and CDR regions are derived from mouse germline immunoglobulin sequences. Furthermore, if the antibody contains a constant region, the constant region also is derived from mouse germline immunoglobulin sequences. The mouse antibodies of the disclosure can include amino acid residues not encoded by mouse germline immunoglobulin sequences (e.g., mutations introduced by random or site-specific mutagenesis in vitro or by somatic mutation in vivo). However, the term “mouse antibody”, as used herein, is not intended to include antibodies in which CDR sequences derived from the germline of another mammalian species have been grafted onto mouse framework sequences.

The term “chimeric antibody” refers to an antibody made by combining genetic material from a nonhuman source with genetic material from a human being. Or more generally, a chimeric antibody is an antibody having genetic material from a certain species with genetic material from another species.

The term “humanized antibody”, as used herein, refers to an antibody from non-human species whose protein sequences have been modified to increase similarity to antibody variants produced naturally in humans.

The phrases “an antibody recognizing an antigen” and “an antibody specific for an antigen” are used interchangeably herein with the term “an antibody which binds specifically to an antigen.”

As used herein, an antibody that “specifically binds to human TIGIT” is intended to refer to an antibody that binds to human TIGIT protein (and possibly a TIGIT protein from one or more non-human species) but does not substantially bind to non-TIGIT proteins. Preferably, the antibody binds to human TIGIT protein with “high affinity”, namely with a K_{D} of 5.0×10⁻⁸ M or less, more preferably 5.0×10⁻⁸ M or less, and more preferably 1.0×10⁻⁹M or less.

The term “does not substantially bind” to a protein or cells, as used herein, means does not bind or does not bind with a high affinity to the protein or cells, i.e. binds to the protein or cells with a K_{D} of 1.0×10⁻⁶ M or more, more preferably 1.0×10⁻⁵ M or more, more preferably 1.0×10⁻⁴ M or more, more preferably 1.0×10⁻² M or more, even more preferably 1.0×10⁻² M or more.

The term “high affinity” for an IgG antibody refers to an antibody having a K_{D} of 1.0×10⁻⁶ M or less, more preferably 5.0×10⁻⁸ M or less, even more preferably 1.0×10⁻⁸ M or less, even more preferably 5.0×10⁻⁹ M or less and even more preferably 1.0×10⁻⁹ M or less for a target antigen. However, “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 10⁻⁸ M or less.

The term “K_{assoc}” or “Kₐ”, as used herein, is intended to refer to the association rate of a particular antibody-antigen interaction, whereas the term “K_{dis}” or “K_{d}”, as used herein, is intended to refer to the dissociation rate of a particular antibody-antigen interaction. The term “K_{D}”, as used herein, is intended to refer to the dissociation constant, which is obtained from the ratio of Kₐ to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The term “EC₅₀”, also known as half maximal effective concentration, refers to the concentration of an antibody which induces a response halfway between the baseline and maximum after a specified exposure time.

The term “antibody dependent cellular cytotoxicity”, “antibody dependent cell-mediated cytotoxicity” or “ADCC” refers to a mechanism of cell-mediated immune defense whereby an effector cell of the immune system actively lyses a target cell bound by antibodies.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “antagonistic TIGIT antibody” or “antagonistic anti-TIGIT antibody” refers to an anti-TIGIT antibody that binds to TIGIT and blocks TIGIT signaling induced by TIGIT's interaction with its ligands such as PVR. The antagonistic anti-TIGIT antibody may promote T cell activation and cytokine release, and enhance immunity, and thus can be used to treat e.g., cancers and chronic infections.

The term “therapeutically effective amount” means an amount of the antibody or antigen binding portion thereof of the present disclosure sufficient to prevent or ameliorate the symptoms associated with a disease or condition (such as a cancer) and/or lessen the severity of the disease or condition. A therapeutically effective amount is understood to be in context of the condition being treated, where the actual effective amount is readily discerned by those of skill in the art.

Various aspects of the disclosure are described in further detail in the following subsections.

The exemplary antibodies, or antigen binding portions thereof, of the disclosure specifically bind to human and monkey TIGIT proteins with high binding capabilities that are similar to or higher than those of Tiragolumab and Etigilimab. The antibodies or antigen binding portions thereof of the disclosure may also block TIGIT-PVR binding or interaction, and the blocking activities are comparable to those of Tiragolumab and Etigilimab.

More importantly, the antibodies or antigen binding portions thereof of the disclosure have comparable, if not higher, T cell activating capabilities and in vivo anti-tumor activities.

Preferred antibodies or antigen binding portions thereof of the disclosure are monoclonal. Additionally, the antibodies or antigen binding portions thereof can be, for example, mouse, chimeric or humanized.

The exemplary antibodies or antigen binding portions thereof of the disclosure are structurally and chemically characterized below. The sequence ID numbers of their heavy chain and light chain variable regions and CDRs are summarized in Table 1, some antibodies or antigen binding portions thereof share the same heavy/light chain variable regions.

TABLE 1
Amino acid sequence ID NOs. of heavy/light chain variable regions and CDRs

| mAb/SEQ ID NO. | HV-CDR1 | HV-CDR2 | HV-CDR3 | HV | LV-CDR1 | LV-CDR2 | LV-CDR3 | LV |
|---|---|---|---|---|---|---|---|---|
| Mouse and chimeric 70E11 | 1 | 2 | 3 | 13 | 4 | 5 | 6 | 24 |
| 70E11-VH1VL1 | 1 | 2 | 3 | 14 | 4 | 5 | 6 | 25 |
| 70E11-VH2VL2 | 1 | 2 | 3 | 15 | 4 | 5 | 6 | 26 |
| 70E11-VH2VL3 | 1 | 2 | 3 | 15 | 4 | 5 | 6 | 27 |
| 70E11-VH2VL4 | 1 | 2 | 3 | 15 | 4 | 5 | 6 | 28 |
| 70E11-VH3VL2 | 1 | 2 | 3 | 16 | 4 | 5 | 6 | 26 |
| 70E11-VH3VL3 | 1 | 2 | 3 | 16 | 4 | 5 | 6 | 27 |
| 70E11-VH3VL4 | 1 | 2 | 3 | 16 | 4 | 5 | 6 | 28 |
| 70E11-VH4VL2 | 1 | 2 | 3 | 17 | 4 | 5 | 6 | 26 |
| 70E11-VH4VL3 | 1 | 2 | 3 | 17 | 4 | 5 | 6 | 27 |
| 70E11-VH4VL4 | 1 | 2 | 3 | 17 | 4 | 5 | 6 | 28 |
| 70E11-VH5VL2 | 1 | 2 | 3 | 18 | 4 | 5 | 6 | 26 |
| 70E11-VH5VL3 | 1 | 2 | 3 | 18 | 4 | 5 | 6 | 27 |
| 70E11-VH5VL4 | 1 | 2 | 3 | 18 | 4 | 5 | 6 | 28 |
| Mouse and chimeric 149G11 | 7 | 8 | 9 | 19 | 10 | 11 | 12 | 29 |
| 149G11-VH1VL1 | 7 | 8 | 9 | 20 | 10 | 11 | 12 | 30 |
| 149G11-VH2VL2 | 7 | 8 | 9 | 21 | 10 | 11 | 12 | 31 |
| 149G11-VH2VL3 | 7 | 8 | 9 | 21 | 10 | 11 | 12 | 32 |
| 149G11-VH3VL2 | 7 | 8 | 9 | 22 | 10 | 11 | 12 | 31 |
| 149G11-VH3VL3 | 7 | 8 | 9 | 22 | 10 | 11 | 12 | 32 |
| 149G11-VH4VL2 | 7 | 8 | 9 | 23 | 10 | 11 | 12 | 31 |
| 149G11-VH4VL3 | 7 | 8 | 9 | 23 | 10 | 11 | 12 | 32 |

The heavy chain variable region CDRs and the light chain variable region CDRs in Table 1 have been defined by the Kabat numbering system. However, as is well known in the art, CDR regions can also be determined by other systems such as Chothia, IMGT, AbM, or Contact numbering system/method, based on heavy chain/light chain variable region sequences.

The antibodies of the disclosure may comprise heavy chain constant regions, such as IgG1 constant regions, e.g., human IgG1 constant region having the amino acid sequence of SEQ ID NO: 33, or a functional fragment thereof. The antibodies of the disclosure may comprise light chain constant regions, such as kappa light chain constant regions, e.g., human kappa constant region having the amino acid sequence of SEQ ID NO: 34, or a functional fragment thereof.

The V_{H} and V_{L} sequences (or CDR sequences) of other anti-TIGIT antibodies which bind to human TIGIT can be “mixed and matched” with the V_{H} and V_{L} sequences (or CDR sequences) of the anti-TIGIT antibody of the present disclosure. Preferably, when V_{H} and V_{L} chains (or the CDRs within such chains) are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one embodiment, an antibody of the disclosure, or an antigen binding portion thereof, comprises:

(a) a heavy chain variable region comprising an amino acid sequence listed above in Table 1; and
(b) a light chain variable region comprising an amino acid sequence listed above in Table 1, or the V_{L} of another anti-TIGIT antibody, wherein the antibody specifically binds human TIGIT.

In another embodiment, an antibody of the disclosure, or an antigen binding portion thereof, comprises:

(a) the CDR1, CDR2, and CDR3 regions of the heavy chain variable region listed above in Table 1; and
(b) the CDR1, CDR2, and CDR3 regions of the light chain variable region listed above in Table 1 or the CDRs of another anti-TIGIT antibody, wherein the antibody specifically binds human TIGIT.

In yet another embodiment, the antibody, or antigen binding portion thereof, includes the heavy chain variable CDR2 region of anti-TIGIT antibody combined with CDRs of other antibodies which bind human TIGIT, e.g., CDR1 and/or CDR3 from the heavy chain variable region, and/or CDR1, CDR2, and/or CDR3 from the light chain variable region of a different anti-TIGIT antibody.

In addition, it is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, e.g., Klimka et al., British J. of Cancer 83(2):252-260 (2000); Beiboer et al., J. Mol. Biol. 296:833-849 (2000); Rader et al., Proc. Natl. Acad. Sci. U.S.A. 95:8910-8915 (1998); Barbas et al., J. Am. Chem. Soc. 116:2161-2162 (1994); Barbas et al., Proc. Natl. Acad. Sci. U.S.A. 92:2529-2533 (1995); Ditzel et al., J. Immunol. 157:739-749 (1996); Berezov et al., BIAjournal 8: Scientific Review 8 (2001); Igarashi et al., J. Biochem (Tokyo) 117:452-7 (1995); Bourgeois et al., J. Virol 72:807-10 (1998); Levi et al., Proc. Natl. Acad. Sci. U.S.A. 90:4374-8 (1993); Polymenis and Stoller, J. Immunol. 152:5218-5329 (1994) and Xu and Davis, Immunity 13:37-45 (2000). See also, U.S. Pat. Nos. 6,951,646; 6,914,128; 6,090,382; 6,818,216; 6,156,313; 6,827,925; 5,833,943; 5,762,905 and 5,760,185. Each of these references is hereby incorporated by reference in its entirety.

Accordingly, in another embodiment, antibodies of the disclosure comprise the CDR2 of the heavy chain variable region of the anti-TIGIT antibody and at least the CDR3 of the heavy and/or light chain variable region of the anti-TIGIT antibody, or the CDR3 of the heavy and/or light chain variable region of another anti-TIGIT antibody, wherein the antibody is capable of specifically binding to human TIGIT. These antibodies preferably (a) compete for binding with TIGIT; (b) retain the functional characteristics; (c) bind to the same epitope; and/or (d) have a similar binding affinity as the anti-TIGIT antibody of the present disclosure. In yet another embodiment, the antibodies further may comprise the CDR2 of the light chain variable region of the anti-TIGIT antibody, or the CDR2 of the light chain variable region of another anti-TIGIT antibody, wherein the antibody is capable of specifically binding to human TIGIT. In another embodiment, the antibodies of the disclosure may include the CDR1 of the heavy and/or light chain variable region of the anti-TIGIT antibody, or the CDR1 of the heavy and/or light chain variable region of another anti-TIGIT antibody, wherein the antibody is capable of specifically binding to human TIGIT.

In another embodiment, an antibody of the disclosure comprises a heavy and/or light chain variable region sequences of CDR1, CDR2 and CDR3 sequences which differ from those of the anti-TIGIT antibodies of the present disclosure by one or more conservative modifications. It is understood in the art that certain conservative sequence modification can be made which do not remove antigen binding. See, e.g., Brummell et al., (1993) Biochem 32:1180-8; de Wildt et al., (1997) Prot. Eng. 10:835-41; Komissarov et al., (1997) J. Biol. Chem. 272:26864-26870; Hall et al., (1992) J. Immunol. 149:1605-12; Kelley and O'Connell (1993) Biochem. 32: 6862-35; Adib-Conquy et al., (1998) Int. Immunol. 10:341-6 and Beers et al., (2000) Clin. Can. Res. 6:2835-43.

Accordingly, in one embodiment, the antibody comprises a heavy chain variable region comprising CDR1, CDR2, and CDR3 sequences and/or a light chain variable region comprising CDR1, CDR2, and CDR3 sequences, wherein:

(a) the heavy chain variable region CDR1 sequence comprises a sequence listed in Table 1 above, and/or conservative modifications thereof; and/or
(b) the heavy chain variable region CDR2 sequence comprises a sequence listed in Table 1 above, and/or conservative modifications thereof; and/or
(c) the heavy chain variable region CDR3 sequence comprises a sequence listed in Table 1 above, and conservative modifications thereof; and/or
(d) the light chain variable region CDR1, and/or CDR2, and/or CDR3 sequences comprise the sequence(s) listed in Table 1 above; and/or conservative modifications thereof; and
(e) the antibody specifically binds human TIGIT.

The antibody of the present disclosure possesses one or more of the following functional properties described above, such as high affinity binding to human TIGIT, and the ability to induce antibody dependent cell mediated cytotoxicity (ADCC) or complement dependent cytotoxicity (CDC) to TIGIT positive cells.

In various embodiments, the antibody or antigen binding portion thereof, of the disclosure can be, for example, mouse, chimeric, human, or humanized.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the disclosure can be replaced with other amino acid residues from the same side chain family and the altered antibody can be tested for retained function (i.e., the functions set forth above) using the functional assays described herein.

The antibody of the disclosure can be prepared using an antibody having one or more of the V_{H}/V_{L} sequences of the anti-TIGIT antibody of the present disclosure as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Accordingly, another embodiment of the disclosure pertains to an isolated monoclonal antibody, or antigen binding portion thereof, comprising a heavy chain variable region comprising CDR1, CDR2, and CDR3 sequences comprising the sequences of the present disclosure, as described above, and/or a light chain variable region comprising CDR1, CDR2, and CDR3 sequences comprising the sequences of the present disclosure, as described above. While these antibodies contain the V_{H} and V_{L} CDR sequences of the monoclonal antibody of the present disclosure, they can contain different framework sequences.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., (1991), cited supra; Tomlinson et al., (1992) J. Mol. Biol. 227:776-798; and Cox et al., (1994) Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank Accession Nos.: 1-69 (NG-0010109, NT-024637 & BC070333), 3-33 (NG-0010109 & NT-024637) and 3-7 (NG-0010109 & NT-024637). As another example, the following heavy chain germline sequences found in the HCo12 HuMAb mouse are available in the accompanying Genbank Accession Nos.: 1-69 (NG-0010109, NT-024637 & BC070333), 5-51 (NG-0010109 & NT-024637), 4-34 (NG-0010109 & NT-024637), 3-30.3 (CAJ556644) & 3-23 (AJ406678).

Antibody protein sequences are compared against a compiled protein sequence database using one of the sequence similarity searching methods called the Gapped BLAST (Altschul et al., (1997), supra), which is well known to those skilled in the art.

Preferred framework sequences for use in the antibodies of the disclosure are those that are structurally similar to the framework sequences used by antibodies of the disclosure. The V_{H} CDR1, CDR2, and CDR3 sequences can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced. The mutations can be amino acid substitutions, additions or deletions, but are preferably substitutions. Moreover, typically no more than one, two, three, four or five residues within a CDR region are altered.

Accordingly, in another embodiment, the disclosure provides isolated anti-TIGIT monoclonal antibodies, or antigen binding portions thereof, comprising a heavy chain variable region comprising: (a) a V_{H} CDR1 region comprising the sequence of the present disclosure, or an amino acid sequence having one, two, three, four or five amino acid substitutions, deletions or additions; (b) a V_{H} CDR2 region comprising the sequence of the present disclosure, or an amino acid sequence having one, two, three, four or five amino acid substitutions, deletions or additions; (c) a V_{H} CDR3 region comprising the sequence of the present disclosure, or an amino acid sequence having one, two, three, four or five amino acid substitutions, deletions or additions; (d) a V_{L} CDR1 region comprising the sequence of the present disclosure, or an amino acid sequence having one, two, three, four or five amino acid substitutions, deletions or additions; (e) a V_{L} CDR2 region comprising the sequence of the present disclosure, or an amino acid sequence having one, two, three, four or five amino acid substitutions, deletions or additions; and (f) a V_{L} CDR3 region comprising the sequence of the present disclosure, or an amino acid sequence having one, two, three, four or five amino acid substitutions, deletions or additions.

Engineered antibodies of the disclosure include those in which modifications have been made to framework residues within V_{H} and/or V_{L}, e.g. to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or as an alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the disclosure can be chemically modified (e.g., one or more chemical moieties can be attached to the antibody) or be modified to alter its glycosylation, again to alter one or more functional properties of the antibody.

In one embodiment, the hinge region of C_{H1} is modified in such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of an antibody is modified. For example, a glycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. For example, the cell lines Ms704, Ms705, and Ms709 lack the fucosyltransferase gene, FUT8 (α(1,6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704 and Yamane-Ohnuki et al., (2004) Biotechnol Bioeng 87:614-22). As another example, EP 1,176,195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1,176,195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). Antibodies with a modified glycosylation profile can also be produced in chicken eggs, as described in PCT Publication WO 06/089231. Alternatively, antibodies with a modified glycosylation profile can be produced in plant cells, such as Lemna. Methods for production of antibodies in a plant system are disclosed in the U.S. patent application corresponding to Alston & Bird LLP attorney docket No. 040989/314911, filed on Aug. 11, 2006. PCT Publication WO 99/54342 describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al., (1999) Nat. Biotech. 17:176-180). Alternatively, the fucose residues of the antibody can be cleaved off using a fucosidase enzyme; e.g., the fucosidase α-L-fucosidase removes fucosyl residues from antibodies (Tarentino et al., (1975) Biochem. 14:5516-23).

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See, e.g., EPO 154 316 and EP 0 401 384.

Antibodies of the disclosure can be characterized by their various physical properties, to detect and/or differentiate different classes thereof.

For example, antibodies can contain one or more glycosylation sites in either the light or heavy chain variable region. Such glycosylation sites may result in increased immunogenicity of the antibody or an alteration of the pK of the antibody due to altered antigen binding (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al., (2000) Mol Immunol 37:697-706). Glycosylation has been known to occur at motifs containing an N-X-S/T sequence. In some instances, it is preferred to have an anti-TIGIT antibody that does not contain variable region glycosylation. This can be achieved either by selecting antibodies that do not contain the glycosylation motif in the variable region or by mutating residues within the glycosylation region.

In a preferred embodiment, the antibodies do not contain asparagine isomerism sites. The deamidation of asparagine may occur on N-G or D-G sequences and result in the creation of an isoaspartic acid residue that introduces a kink into the polypeptide chain and decreases its stability (isoaspartic acid effect).

Each antibody will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-TIGIT antibody that contains a pI value that falls in the normal range. This can be achieved either by selecting antibodies with a pI in the normal range or by mutating charged surface residues.

In another aspect, the disclosure provides nucleic acid molecules that encode heavy and/or light chain variable regions, or CDRs, of the antibodies of the disclosure. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a cDNA molecule.

Nucleic acids of the disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of the disclosure include those encoding the V_{H} and V_{L} sequences of the TIGIT monoclonal antibody or the CDRs. Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (Cm, C_{H2} and C_{H3}). The sequences of human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al., (1988) Science 242:423-426; Huston et al., (1988) Proc. Natl. Acad. Sci. USA 85:5879-5883; McCafferty et al., (1990) Nature 348:552-554).

Monoclonal antibodies (mAbs) of the present disclosure can be produced using the well-known somatic cell hybridization (hybridoma) technique of Kohler and Milstein (1975) Nature 256: 495. Other embodiments for producing monoclonal antibodies include viral or oncogenic transformation of B lymphocytes and phage display techniques. Chimeric or humanized antibodies are also well known in the art. See e.g., U.S. Pat. Nos. 4,816,567; 5,225,539; 5,530,101; 5,585,089; 5,693,762 and 6,180,370, the contents of which are specifically incorporated herein by reference in their entirety.

Antibodies of the disclosure also can be produced in a host cell transfectoma using, for example, a combination of recombinant DNA techniques and gene transfection methods as is well known in the art (e.g., Morrison, S. (1985) Science 229:1202). In one embodiment, DNA encoding partial or full-length light and heavy chains obtained by standard molecular biology techniques is inserted into one or more expression vectors such that the genes are operatively linked to transcriptional and translational regulatory sequences. In this context, the term “operatively linked” is intended to mean that an antibody gene is ligated into a vector such that transcriptional and translational control sequences within the vector serve their intended function of regulating the transcription and translation of the antibody gene.

The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody light chain gene and the antibody heavy chain gene can be inserted into the same or separate expression vectors. In preferred embodiments, the variable regions are used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the disclosure include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

The antibody or antigen binding portion thereof of the disclosure may be conjugated to a therapeutic agent to form an immunoconjugate such as an antibody-drug conjugate (ADC). Suitable therapeutic agents include cytotoxins, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. More preferably, the linker is a peptidyl linker such as Val-Cit, Ala-Val, Val-Ala-Val, Lys-Lys, Pro-Val-Gly-Val-Val, Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038,658; WO 07/051,081; WO 07/059,404; WO 08/083,312; and WO 08/103,693; U.S. Patent Publications 20060024317; 20060004081; and 20060247295; the disclosures of which are incorporated herein by reference.

In another aspect, the present disclosure features a bispecific molecule comprising the antibody or antigen binding portion thereof of the disclosure linked to at least one other functional molecule, e.g., another peptide or protein (e.g., another antibody or ligand for a receptor) to generate a bispecific molecule that binds to at least two different binding sites or target molecules. Thus, as used herein, the “bispecific molecule” includes molecules that have three or more specificities.

In an embodiment, a bispecific molecule has, in addition to an anti-Fc binding specificity and an anti-TIGIT binding specificity, a third specificity. The third specificity can be for an anti-enhancement factor (EF), e.g., a molecule that binds to a surface protein involved in cytotoxic activity and thereby increases the immune response against the target cell. For example, the anti-enhancement factor can bind a cytotoxic T-cell (e.g. via CD2, CD3, CD8, CD28, CD4, TIGIT, or ICAM-1) or other immune cell, resulting in an increased immune response against the target cell.

The bispecific molecules may be in many different formats and sizes. At one end of the size spectrum, a bispecific molecule retains the traditional antibody format, except that, instead of having two binding arms of identical specificity, it has two binding arms each having a different specificity. At the other extreme are bispecific molecules consisting of two single-chain antibody fragments (scFv's) linked by a peptide chain, a so-called Bs(scFv) 2 construct. Intermediate-sized bispecific molecules include two different F(ab) fragments linked by a peptidyl linker. Bispecific molecules of these and other formats can be prepared by genetic engineering, somatic hybridization, or chemical methods. See, e.g., Kufer et al, cited supra; Cao and Suresh, Bioconjugate Chemistry, 9 (6), 635-644 (1998); and van Spriel et al., Immunology Today, 21 (8), 391-397 (2000), and the references cited therein.

The disclosure provides a chimeric antigen receptor comprising an anti-TIGIT single chain variable fragment (scFv) comprising the heavy and light chain variable regions and/or CDRs of the disclosure.

The chimeric antigen receptor may comprise (a) an extracellular antigen recognition domain containing the anti-TIGIT scFv, (b) a transmembrane domain, and (c) an intracellular signaling domain.

An oncolytic virus preferentially infects and kills cancer cells. The antibody or antigen binding portion thereof of the disclosure may be used in conjunction with the oncolytic virus. Alternatively, an oncolytic virus encoding the antibody or antigen binding portion thereof of the disclosure can be introduced into human body.

In another aspect, the present disclosure provides a pharmaceutical composition comprising the antibody or antigen binding portion thereof, the immunoconjugate, the bispecific molecule, the immune cell carrying the chimeric antigen receptor, the oncolytic virus, the nucleic acid molecule, the expression vector, and/or the host cell of the present disclosure formulated together with a pharmaceutically acceptable carrier. The composition may optionally contain one or more additional pharmaceutically active ingredients, such as an anti-tumor agent, an anti-infective agent, or an agent for immunity enhancement. The pharmaceutical composition of the disclosure may be administered in a combination therapy with, for example, an anti-tumor agent, an anti-infective agent, or an agent for immunity enhancement.

The pharmaceutical composition may comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

The pharmaceutical composition may be in the form of a sterile aqueous solution or dispersion. The pharmaceutical composition may also be formulated in a microemulsion, liposome, or other ordered structure suitable for high drug concentration.

The amount of the active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about 99% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the antibody or antigen binding portion thereof, the dosage may range from about 0.0001 to 100 mg/kg body weight. An exemplary treatment regime entails administration once per week. Preferred dosage regimens for an anti-TIGIT antibody of the disclosure include intravenous administration.

A “therapeutically effective dosage” of an anti-TIGIT antibody of the disclosure preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 20%, more preferably by at least about 40%, even more preferably by at least about 60%, and still more preferably by at least about 80% relative to untreated subjects. A therapeutically effective amount of a therapeutic antibody can decrease tumor size, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the monoclonal antibodies of the disclosure can be formulated to ensure proper distribution in vivo. For example, to ensure that the therapeutic antibody of the disclosure cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331; V. V. Ranade (1989) J. Clin. Pharmacol. 29:685; Umezawa et al., (1988) Biochem. Biophys. Res. Commun. 153:1038; Bloeman et al., (1995) FEBS Lett. 357:140; M. Owais et al., (1995) Antimicrob. Agents Chemother. 39:180; Briscoe et al., (1995) Am. J. Physiol. 1233:134; Schreier et al., (1994) J. Biol. Chem. 269:9090; Keinanen and Laukkanen (1994) FEBS Lett. 346:123; and Killion and Fidler (1994) Immunomethods 4:273.

The pharmaceutical composition of the disclosure may have numerous in vitro and in vivo utilities involving, for example, treatment and/or prevention of cancers and infectious diseases. The pharmaceutical composition of the disclosure may be administered to human subjects, to inhibit tumor growth, or eliminate pathogens.

Given the ability of anti-TIGIT antibodies of the disclosure to inhibit proliferation and survival of cancer cells, the disclosure provides methods for inhibiting growth of tumor cells in a subject comprising administering to the subject the pharmaceutical composition of the disclosure such that growth of the tumor is inhibited in the subject. Non-limiting examples of tumors that can be treated by antibodies of the disclosure include, but not limited to, liver cancer, rectal cancer, endometrial cancer, pancreas cancer, non-small cell lung cancer, multiple myeloma, and melanoma, original and/or metastatic. Additionally, refractory or recurrent malignancies may be inhibited using the pharmaceutical composition of the disclosure.

In another aspect, as the pharmaceutical composition of the disclosure may reduce or eliminate the pathogens, the disclosure provides a method for treating an infectious disease in a subject in need thereof, comprising administering to the subject the pharmaceutical composition of the disclosure. The infectious disease may be caused by viral, bacterial, fungal, or mycoplasma infection. The infectious disease may be a chronic HIV infection.

In another aspect, the disclosure provides methods of combination therapy in which the pharmaceutical composition of the disclosure is co-administered with one or more additional antibodies or non-antibody agents that are effective in inhibiting tumor growth in a subject. In one embodiment, the disclosure provides a method for inhibiting tumor growth in a subject comprising administering to the subject the pharmaceutical composition of the disclosure with one or more additional antibodies, such as an anti-VISTA antibody, an anti-LAG-3 antibody, an anti-PD-L1 antibody, and anti-PD-1 antibody and/or an anti-CTLA-4 antibody. The pharmaceutical composition of the disclosure may be used in combination with a chemotherapeutic agent, which is toxic to cells. Other therapies that may be combined with anti-TIGIT antibody includes, but not limited to, interleukin-2 (IL-2) administration, radiation, surgery, or hormone deprivation. In certain embodiments, the subject is human.

The pharmaceutical composition of the disclosure may be used in combination with one or more other antibodies or non-antibody agents, to effectively reduce or eliminate pathogens in a subject, such as viruses, bacteria, fungi, or mycoplasmas. For example, the pharmaceutical composition of the disclosure may be used with an anti-infectious agent, including, but not limited to, an anti-virus agent, an anti-bacterial agent, an anti-fungal agent, and an anti-mycoplasma agent.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1 Construction of HEK293A Cell Lines Stably Expressing Human TIGIT, Monkey TIGIT, Mouse TIGIT, or Human PVR

Cell lines respectively stably overexpressing human TIGIT, monkey TIGIT, mouse TIGIT, and human PVR were constructed using HEK293A cells (Cobioer, NJ, CN). Briefly, cDNA sequences respectively encoding human TIGIT, monkey TIGIT, mouse TIGIT and human PVR (amino acid sequences set forth in SEQ ID NOs: 35, 36, 37 and 38) were synthesized, and then subcloned into pLV-EGFP(2A)-Puro vectors (Inovogen Biotech, BJ, CN). Lentiviruses were generated in HEK-293T cells (Cobioer, NJ, CN) by cotransfection of pLV-EGFP(2A)-Puro-TIGIT/pLV-EGFP(2A)-Puro-PVR, psPAX and pMD2.G plasmids, according to the instruction in Lipofectamine 3000 kit (Thermo Fisher Scientific, US). Three days post cotransfection, the lentiviruses were harvested from the cell culture medium (DMEM medium (Cat #: SH30022.01, Gibco) with 10% FBS (Cat #: FND500, Excell)) of the HEK-293T cells. Finally, HEK293A cells (Cobioer, NJ, CN) were infected with the lentiviruses to generate HEK293A cell lines stably expressing human, monkey or mouse TIGIT, namely HEK293A/human TIGIT, HEK293A/monkey TIGIT or HEK293A/mouse TIGIT cells; A549 cells (Cobioer, NJ, CN) were infected with the lentiviruses to generate a A549 cell line stably expressing human PVR, referred to as A549/human PVR cells. Transfected HEK293A and A549 cells were then respectively cultured in medium (DMEM+10% FBS) containing 0.2 μg/ml puromycin (Cat #: A11138-03, Gibco) for 7 days. The expressions of human TIGIT and cynomolgus TIGIT were confirmed by FACS using a commercially available anti-TIGIT antibody (PE-anti-human TIGIT, Cat #: 372703, Biolegend, US). Similarly, the expression of mouse VISTA was confirmed by FACS using a commercially available anti-mouse TIGIT antibody (PE-anti-mouse TIGIT, Cat #: 622205, Biolegend, US). The expression of human PVR was confirmed by FACS using a commercially available anti-PVR antibody (PE-anti-human PVR, Cat #: 566718, BD, US).

### Example 2 Generation of Hybridoma Cell Lines Producing Monoclonal Mouse Antibodies Against Human TIGIT

Murine anti-human TIGIT monoclonal antibodies (mAbs) were generated using the conventional hybridoma fusion technology with some modifications.

### Immunization

Ten BALB/c mice (Beijing Vital River Laboratory Animal Technology Co., Ltd, Beijing, CN) were injected with recombinant human TIGIT (ECD)-hFc (Cat #: 10917-H02H, Sino Biological, CN) and recombinant cynomolgus TIGIT (ECD)-hFc (Cat #: TIT-05254, Sino Biological, CN) following the scheme in Table 2 below. The human TIGIT (ECD)-hFc and cynomolgus TIGIT (ECD)-hFc were emulsified by sonication with an equal volume of Complete Freund's Adjuvant (Cat #: F5881-10*10ML, SIGMA, US), Incomplete Freund's Adjuvant (Cat #: F5506-6*10ML, SIGMA, US), or PBS.

TABLE 2
Immunization scheme

| | Primary | 1st Boost | 2nd Boost | 3rd Boost | Final Boost |
|---|---|---|---|---|---|
| Day | 0 | 14 | 28 | 42 | 56 |
| Immunogen | Human TIGIT | Human TIGIT | Cynomolgus | Human TIGIT | Cynomolgus |
| and dose | (ECD)-hFc | (ECD)-hFc | TIGIT (ECD)-hFc | (ECD)-hFc | TIGIT (ECD)-hFc |
| | (50 μg/mouse) | (50 μg/mouse) | (50 μg/mouse) | (50 μg/mouse) | (25 μg/mouse) + |
| | | | | | Human TIGIT |
| | | | | | (ECD)-hFc |
| | | | | | (25 μg/mouse) |
| Adjuvant | Complete | Incomplete | Incomplete | Incomplete | PBS |
| | Freund's | Freund's | Freund's | Freund's | |
| Immunization | Intraperitoneal | i.p. | i.p. | i.p. | Intravenous |
| | injection (i.p.) | | | | injection (i.v.) |

One week after each boost, 50 μl murine serum from each mouse was subject to titer determination by ELISA using the recombinant human TIGIT (ECD)-his (Cat #: 10917-H08H, Sino Biological, CN) and cyno TIGIT (ECD)-hFc (Cat #: TIT-05254, Sino Biological, CN). Titer determination was also done by FACS using HEK293A cells overexpressing human TIGIT, cynomolgus TIGIT or mouse TIGIT as prepared in Example 1.

Based on the ELISA and FACS analysis results after the final boost, 8 mice with highest serum titers were selected for hybridoma cell line generation.

### Generation of Hybridoma Cell Lines

Hybridoma cell lines were generated using the conventional hybridoma fusion technology with minor modifications.

Four days after the final boost, mice were sacrificed, and spleens were collected and prepared as single cell suspensions in PBS. The spleenocytes were washed for three times with DMEM medium (Cat #: SH30243.01B, Hyclone, US). Viable myeloma cells SP2/0 (CRL-1581, ATCC, US) at the log-phase were mixed with the murine spleenocytes at a ratio of 1:4. The cells were then washed twice and then cell fusion was performed with PEG (Cat #: P7181, Sigma, US). The post-fusion cells were washed with DMEM medium for three times and suspended in cell growth medium (RPMI medium 1640 (Cat #: C22400500CP, Gibco)) supplemented with 10% FBS and 1×HAT (H0262, Sigma). The cell suspensions were plated onto 96 well cell culture plates, 200 μl per well, containing about 5×10⁴ cells, and incubated in a 37° C. humidified 5% CO₂ incubator for 7 days. Then, the growth medium was replaced by fresh one supplemented with 10% FBS and 1×HAT. Two to three days later, cell culture supernatants were collected for hybridoma cell screening by ELISA and FACS.

### Screening of Hybridoma Cell Lines by ELISA

High-throughput ELISA binding assay was performed to screen for hybridoma clones producing monoclonal antibodies binding to human TIGIT, using human TIGIT (ECD)-his (Cat #: 10917-H08H, Sino Biological, CN). The hybridoma clones producing antibodies binding to human TIGIT were further tested for their abilities to cross-react with cynomolgus TIGIT using cynomolgus TIGIT (ECD)-hFc (Cat #: TIT-05254, Sino Biological, CN).

With the ELISA assays, 85 hybridoma clones were identified to have specific binding to both human and monkey TIGIT.

### Screening of Hybridoma Cell Lines by FACS

The 85 hybridoma clones were further tested for their binding capabilities to human, cynomolgus and mouse TIGITs expressed on HEK293A cells, using the HEK293A/human TIGIT cells, HEK293A/monkey TIGIT cells and HEK293A/mouse TIGIT cells as prepared in Example 1.

Based on the FACS screening, 58 positive clones were obtained that displayed high binding capabilities to both HEK293A/human TIGIT cells and HEK293A/monkey TIGIT cells but no binding capability to HEK293A/mouse TIGIT cells.

### Subcloning of Hybridoma Clones Producing Anti-TIGIT Antibodies

The 58 hybridoma clones were subject to 2 rounds of subcloning. During the subcloning, multiple subclones (n>3) from each parent clone were selected and tested by ELISA and FACS assays as described above. The subclones selected through this process were defined as hybridoma cells producing monoclonal antibodies. Finally, 28 subclones (one subclone from each parent clone) having high binding capabilities to both human and monkey TIGIT were obtained.

### Example 3 Purification of Mouse Anti-TIGIT Monoclonal Antibodies

Twenty clones, out of the 28, producing antibodies with relatively high binding capabilities to human and monkey TIGITs were further characterized. The monoclonal antibodies from the 20 clones were purified. Briefly, the hybridoma cells of each clone were grown in T175 cell culture flasks each having 100 ml fresh serum-free medium (Cat #: 12045-076, Gibco, US) with 1% HT supplement (Cat #: 11067-030, Gibco, US). Cells were cultured for 10 days in an incubator with 5% CO₂ at 37° C. Cell cultures were collected, followed by centrifugation at 3500 rpm for 5 minutes and then subject to filtration using a 0.22 μm filter membrane to remove cell debris. Monoclonal mouse antibodies were then purified and enriched using pre-equilibrated Protein-A affinity columns (Cat #: 17040501, GE, US) and eluted with elution buffer (20 mM citric acid, pH3.0-pH3.5). Then, antibodies were kept in PBS buffer (pH 7.0), and determined for concentrations using a NanoDrop instrument.

The isotypes of the purified antibodies were determined by using Rapid Antibody Isotyping Kit with Kappa and Lambda-Mouse (Cat #: 26179, Thermal, US) and Mouse Monoclonal Antibody Isotyping Reagents (Cat #: IS02-1KT, Sigma, US), following the manufacturer's manuals.

Most clones, including 70E11 and 149G11, produced mIgG1/kappa antibodies, while a few others produced mIgG2a/kappa antibodies. The expression titers for clone 70E11 and 149G11 were 1.2 mg/L and 24.2 mg/L, respectively.

### Example 4 Purified Mouse Anti-TIGIT Monoclonal Antibodies Bound to Human and Monkey TIGIT

Purified mouse anti-TIGIT monoclonal antibodies were characterized by ELISA assays for their binding capabilities to recombinant human or monkey TIGIT proteins.

ELISA plates were coated with 500 ng/ml human TIGIT (ECD)-his (Cat #: 10917-H08H, Sino Biological, CN) at 4° C. overnight, 100 μl per well. The wells were blocked with 200 μl blocking buffer (PBS containing 1% BSA, 1% goat serum, and 0.05% Tween20) for 2 h at room temperature, and then 100 μl serially diluted anti-TIGIT antibodies (starting from 40000 ng/ml) were added to each well and incubated for 1 h at room temperature. Plates were washed for 3 times with PBST (PBS+0.05% Tween20), added with Goat-anti-mouse IgG-HRP (Cat #: A9309-1 ml, Sigma, US) diluted 5000×, and incubated for 1 h at room temperature. Plates were incubated with freshly prepared Ultra-TMB (Cat #: 555214, BD, US) for 5 minutes at room temperature for color development.

Species-cross-reactivities of the 20 TIGIT monoclonal antibodies to monkey TIGIT were further assessed by direct ELISA. Briefly, 500 ng/ml monkey TIGIT (ECD)-hFc (Cat #: TIT-05254, Sino Biological, CN) was coated on 96-well ELISA plates, 100 μl per well, followed by incubation with 100 μl serially diluted anti-TIGIT antibodies (starting from 40000 ng/ml). Goat anti-mouse IgG conjugated with HRP (Cat #: A9309-1 ml, Sigma, US) was then used.

Tiragolumab, prepared using the amino acid sequences disclosed in US20170088613A1 with human IgG1/kappa constant regions, or alternatively the sequences of INN 10644_H and INN 10644_L published on http://www.imgtorg/3Dstructure-DB/cgi/details.cgi?pdbcode=10644, was used as a reference antibody.

EC₅₀ values of representative antibodies were summarized in Table 3. The data showed that the antibodies from all 20 clones bound to human and monkey TIGIT but did not cross-react to mouse TIGIT (data not shown).

TABLE 3
Binding capabilities of representative mouse
anti-TIGIT mAbs to human and monkey TIGIT

| | FACS (EC₅₀: M) | |
|---|---|---|
| Antibodies | hTIGIT(ECD)-his | cynoTIGIT(ECD)-hFc |
| Tiragolumab | 1.6E−10 | 1.4E−10 |
| 70E11 | 8.2E−10 | 3.4E−10 |
| 149G11 | 8.6E−10 | 2.0E−8 |

### Example 5 Mouse Anti-TIGIT Monoclonal Antibodies Bound to Human and Monkey TIGITs Expressed on HEK293A Cells

To further determine whether the anti-TIGIT antibodies bound to human, monkey and mouse TIGITs expressed on HEK293A cells, a cell-based binding assay by FACS was performed using the HEK293A cells respectively stably overexpressing human, monkey and mouse TIGITs as generated in Example 1. Briefly, 10⁵ HEK293A cells in 100 μl cell culture medium were seeded onto each well of the 96-well plates followed by 50 μl serially diluted anti-TIGIT antibodies. After incubated at 4° C. for 1 h, plates were washed 3 times with PBST. Then, APC coupled Goat Anti-Mouse IgG (Cat #: 405308, BioLegend, US) diluted 500x was added to the plates. After incubation at 4° C. for 1 h, the plates were washed with PBS for 3 times and then cell fluorescence was monitored using a FACS machine (BD).

EC₅₀ values of representative antibodies were summarized in Table 4 below. The data indicated that all of the mouse anti-TIGIT monoclonal antibodies had high binding capabilities to both human and monkey TIGITs but not to mouse TIGIT.

TABLE 4
Binding capabilities of mouse anti-TIGIT
antibodies to human, monkey and mouse TIGITs

| | FACS (EC₅₀: M) | | |
|---|---|---|---|
| | HEK-293A/ | HEK-293A/ | HEK-293A/ |
| Antibodies | human TIGIT | monkey TIGIT | mouse TIGIT |
| Tiragolumab | 4.4E−10 | 9E−10 | No binding |
| 70E11 | 5.6E−10 | 3.9E−10 | No binding |
| 149G11 | 1.0E−10 | 1.20E−9 | No binding |

### Example 6 Epitope Binning

For epitope binning, a competition ELISA assay was performed. Briefly, 96-well ELISA plates were coated with 0.5 μg/ml human TIGIT (ECD)-His (Cat #: 10917-H08H, Sino Biological, CN) at 4° C. overnight, 100 μl per well. The wells were each blocked with 200 μl blocking buffer (PBS containing 1% BSA, 1% goat serum, and 0.05% Tween20) for 2 h at room temperature. Tiragolumab, Anti-Hel Human IgG1 isotype control (Cat #: LT12031, LifeTein, US) and Etigilimab (prepared using the amino acid sequences disclosed in US20160376365A1 with human IgG1/kappa constant regions) were respectively diluted to 5 μg/mL and added to the plates, 100 μl per well. The plates were incubated for 1 h at room temperature. The ELISA plates were washed for 3 times with PBST, added with the purified antibodies at 1 μg/mL, and then incubated for 1 h at room temperature. The ELISA plates were washed for 3 times with PBST, added with anti-mouse Fc-HRP (Cat #: A9309-1MC, Sigma, US) diluted at 1:20000, and incubated for 1 h at room temperature. The plates were washed for 3 times with PBST and subject to color development using freshly prepared Ultra-TMB (Cat #: TMB-S-003, Huzhou Yingchuang, CN) for 5 minutes at room temperature. Absorbance at 450 nm was measured on a microplate reader (Thermo Multiscan FC).

Among the 20 mouse antibodies, 7 antibodies, including those from clone 70E11 and 149G11, competed with Tiragolumab over epitope binding, indicating that these antibodies bound the same or similar epitopes as Tiragolumab did. None of the antibodies competed with Etigilimab over epitope binding, meaning the antibodies of the disclosure bound to different epitopes compared to Etigilimab.

### Example 7 Mouse Anti-TIGIT Antibodies Inhibited TIGIT-PVR Interaction

PVR (CD155) was reported as the primary ligand for TIGIT. A cell-based blocking assay by FACS was performed to test the antibodies' blocking capabilities on TIGIT-PVR interaction, using the A549 cells stably overexpressing human PVR as generated in Example 1. Briefly, serially diluted anti-TIGIT antibodies were incubated with TIGIT (ECD)-hFc proteins (Cat #: 10917-H02H, Sino Biological, CN) at a final concentration of 5 μg/ml at 37° C. for 1 h. Then, 10⁵ A549/human PVR cells in 100 μl culture medium were seeded onto each well of 96 well plates, to which further added 100 μl of the antibody/TIGIT (ECD)-hFc mixture as prepared above. After incubated at 4° C. for 1 h, plates were washed 3 times with PBST and then added with PE coupled Goat Anti-human IgG (Cat #: PAI-86078, Thermofisher, US) diluted 500×. After incubation at 4° C. for 1 h, the plates were washed with PBST for 3 times and then cell fluorescence was monitored using a FACS machine (BD).

The data showed that 14 out of the 20 antibodies, including 70E11 and 149G11, were able to block TIGIT-PVR interaction. EC₅₀ values of the representative antibodies were summarized in Table 5.

TABLE 5
Blocking capabilities of mouse anti-TIGIT
antibodies on TIGIT-PVR interaction

| Antibodies | Blocking assay EC₅₀ (M) |
|---|---|
| Tiragolumab | 4.2E−08 |
| 70E11 | 4.3E−08 |
| 149GH | 4.1E−08 |

### Example 8 Mouse Anti-TIGIT Antibodies Activated T Cells

The effects of the mouse anti-TIGIT antibodies on T cell activities were studied by a T cell function assay.

Briefly, PBMCs were collected from blood samples of healthy human donors by density gradient centrifugation, and re-suspended on RPMI1640 medium. CD4⁺ T cells were isolated from the PBMCs using Invitrogen Dynabeads Untouched Human CD4⁺ T cells isolation kit (Cat #: 11346D, Thermal Fisher Scientific, US) according to the manufacturer's instructions. CD4⁺ T cells were re-suspended in complete RPMI culture medium (90% RPMI medium+10% BSA) at the cell density of 1.0×10⁶/ml. Dynabeads Human T-Activator CD3/CD28 beads (Cat #: 11132D, Gibco, US) were added to and incubated with the T cells at 37° C. under 5% CO₂ for 10 days, to activate the T cells.

The CD4⁺ T cells as cultured above were collected and washed with RPIM medium for 3 times, and the cell density was adjusted to 2×10⁵ viable cells/ml. Plates were pre-coated with 0.25 μg/ml anti-CD3 antibodies (OKT3, Cat #: GMP-10977-H001, Sino Biological, CN), 50 μl per well, and 0.25 μg/ml PVR-hFc proteins (Cat: 10109-H02H, Sino Biological, CN), 50 μl per well, overnight at 4° C. The plates were washed with PBS for 3 times and blocked with PBS containing 1% BSA for 90 minutes at 37° C. The plates were washed with PBS for 3 times, added with 150 μl CD4⁺ T cell suspensions prepared above and 50 μl anti-TIGIT antibodies (at a final concentration of 50 μg/ml or serially diluted), and incubated at 37° C. for 3 days in an incubator. Following the manufacture's manuals, 100 μl cell culture supernatant from each well was subject to IFN-γ concentration determination by ELISA (Cat #: SIF50, R&D, US). The assay was done in triplicate.

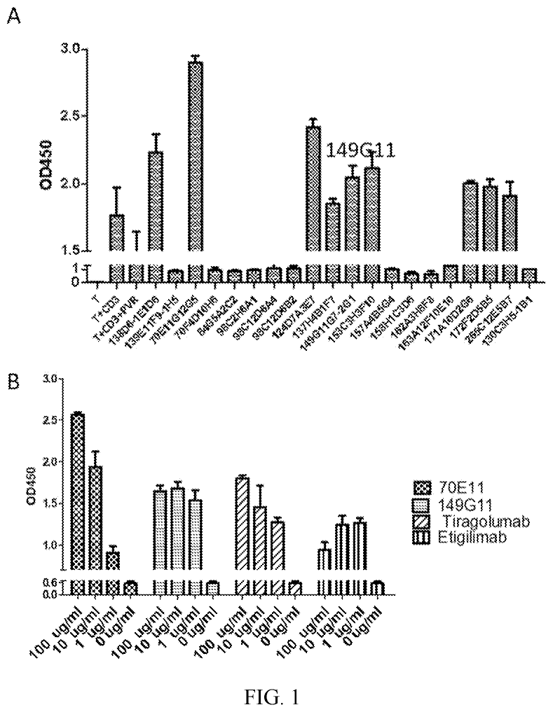

As shown in FIG. 1(A), 9 out of 20 antibodies increased T cell activities as measured by IFN-γ secretion, wherein 70E11 showed the best activation effect, and 149G11 also showed good effect. Further, relative to the anti-HEL control, the antibodies of the disclosure increased IFN-γ release by T cells in a dose dependent manner (FIG. 1(B)). The antibody 70E11 even showed higher activation activity than the positive control at certain concentrations.

### Example 9 Expression and Purification of Chimeric Anti-TIGIT Antibodies

Antibodies 70E11 and 149G11 were selected for further characterization. The heavy/light chain variable region sequences were cloned from the hybridoma cells producing the selected candidate antibodies, using the standard PCR method with a set of degenerated primers as describes in literatures (Juste et al., (2006), Anal Biochem. 349(1):159-161), and then sequenced. The sequences were summarized and listed in Table 1 and Table 10. Expression vectors were constructed by inserting the sequences encoding the variable region sequences plus human IgG1/kappa constant regions (amino acid sequences of heavy and light chain constant regions set forth in SEQ ID NOs: 33 and 34, respectively) between XhoI and BamHI of pCDNA3.1 (Invitrogen, Carlsbad, US).

The expression vectors were PEI transfected into HEK-293F cells (Cobioer, NJ, CN). In specific, HEK-293F cells were cultured in Free Style™ 293 Expression Medium (Cat #: 12338-018, Gibco) and transfected with the expression vectors using polyethyleneimine (PEI) at a DNA: PEI ratio of 1:3, 1.5 μg DNAs per millimeter of cell medium. Transfected HEK-293F cells were cultured in an incubator at 37° C. under 5% CO₂ with shaking at 120 RPM. After 10-12 days, supernatants were harvested and monoclonal antibodies were purified as described in Example 3.

### Example 10 Chimeric Anti-TIGIT Monoclonal Antibodies Bound to Human and Monkey TIGITs

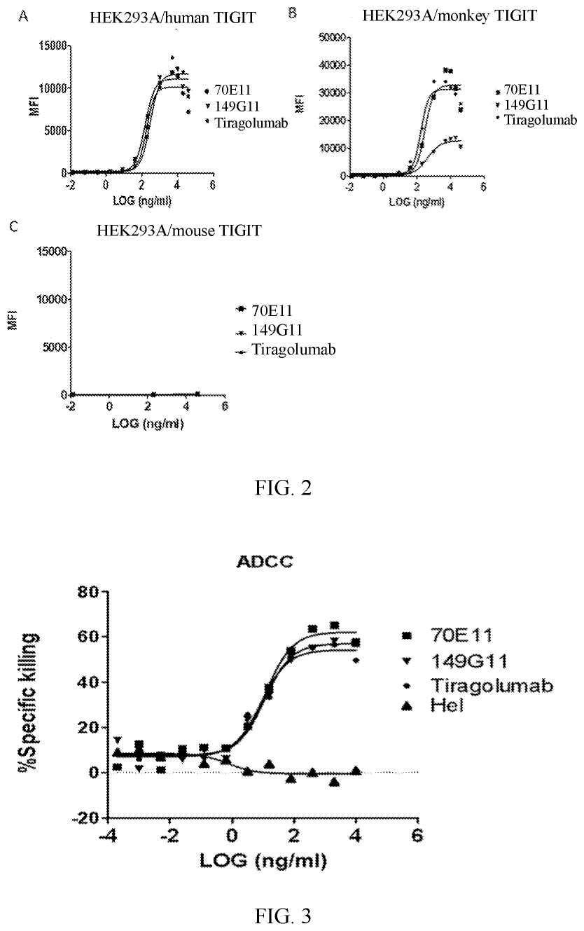

The chimeric anti-TIGIT antibodies were further characterized for their abilities to bind HEK293A/human TIGIT cells, HEK293A/monkey TIGIT cells and HEK293A/mouse TIGIT cells as generated in Example 1, according to the protocol of Example 5. The test results were shown in FIG. 2.

FIG. 2 showed that the chimeric antibodies had high binding capabilities to both human TIGIT (FIG. 2(A)) and monkey TIGIT (FIG. 2(B)), but did not bind mouse TIGIT (FIG. 2 (C)).

### Example 11 ADCCs Induced by Chimeric Anti-TIGIT Antibodies

The chimeric antibodies were analyzed for their capabilities to induce antibody-dependent cellular cytotoxicity (ADCC) against HEK293A/human TIGIT cells. Briefly, HEK293A/human TIGIT cells were generated using the lentivirus transfection system as described in Example 1. The HEK293A/human TIGIT cells and NK92MI-CD16a (as the effector cells, Huabo Bio) were centrifuged at 1200 rpm for 5 minutes. These cells were suspended in the ADCC assay culture medium (MEM medium (Cat #: 12561-056, Gibco)+1% FBS (Cat #: FND500, EX-cell)+1% BSA (Cat #: V900933-1KG, VETEC)), and the cell viability was about 90%. Fifty μ1 HEK293A/human TIGIT cells at a cell density of 4×10⁵/ml, and 50 μl NK92MI-CD16a cells at a cell density of 2×10⁶/ml were added to each well of 96-well plates, with an effector-target ratio at 5:1. The plates were added with antibodies at different concentrations, and the final concentrations of the antibodies were respectively 32000 ng/ml, 6400 ng/ml, 1280 ng/ml, 256 ng/ml, 51.2 ng/ml, 10.24 ng/ml, and 2.048 ng/ml. The plates were incubated at 37° C. for 4 h, and then LDH developing solutions (Cytotoxicity Detection Kit PLUS (LDH), Cat #: 04744926001, Roche) were added, 100 μl per well. The mixtures were kept in dark at room temperature for 20 minutes and then the plates were read by a MD SpectraMax i3. Anti-HEL isotype control (Cat #: LT12031, LifeTein, US) was used a negative control and Tiragolumab as a positive control.

As shown in FIG. 3, the chimeric antibodies 70E11 and 149G11 were both able to induce killing of HEK293A/human TIGIT cells by NK92MI-CD16a, at activities comparable to that of the positive control.

### Example 12 Chimeric Anti-TIGIT Antibodies Inhibited TIGIT-PVR Interaction

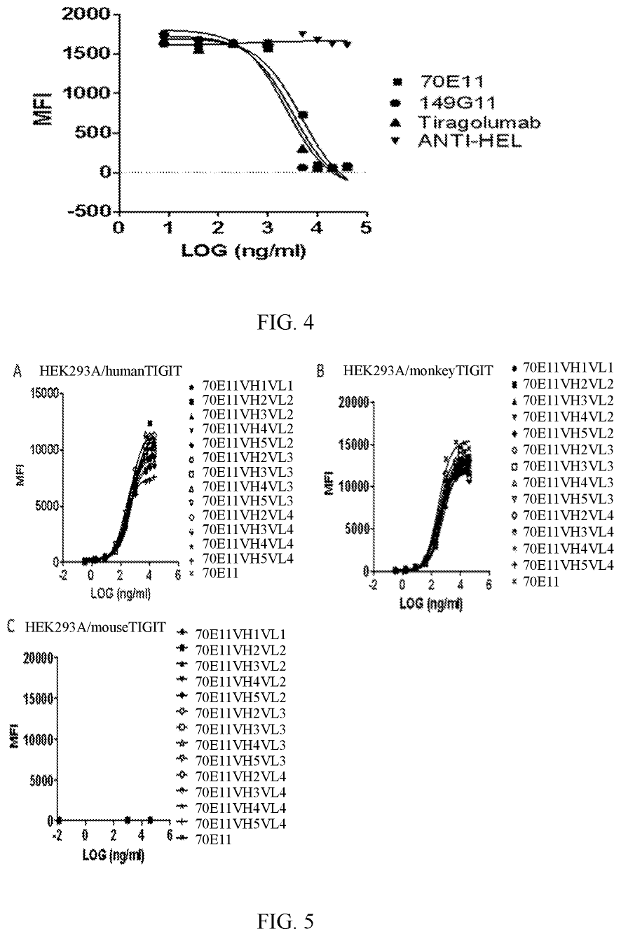

The chimeric anti-TIGIT antibodies were further characterized for their blocking abilities on PVR-TIGIT interaction, using the A549/human PVR cells as generated in Example 1, according to the protocol of Example 7. The test results were shown in FIG. 4, which showed both chimeric antibodies blocked the PVR-TIGIT interaction.

### Example 13 Humanization of Anti-TIGIT Antibodies

Based on the characterizations and assays above, 70E11 and 149G11 were humanized for further investigation. Humanization of the murine antibodies was conducted using the well-established CDR-grafting method (U.S. Pat. No. 5,225,539) as described in detail below.

To select acceptor frameworks for humanization of murine antibodies 70E11 and 149G11, the light and heavy chain variable region sequences of 70E11 and 149G11 were blasted against the human immunoglobulin gene database in NCBI website (http://www.ncbi.nlm.nih.gov/igblast/). Human germline IGVH and IGVK with the highest homology to 70E11 and 149G11 were selected as the acceptors. For 70E11, the human heavy chain acceptor selected was IGHV1-46*01, and the human light chain acceptor selected was IGKV3-20*01. For 149G11, the human heavy chain acceptor selected was IGHV1-46*01, and the human light chain acceptor selected was IGKV4-1*01.

The three dimensional structures were simulated for variable domains of 70E11 and 149G11 in order to identify key framework residues that might be playing important roles in supporting CDR loop structures, based on which back mutations can be designed.

Based on the structural modeling as described above, 8 potential back-mutations (M70L, R72A, M481, T74K, R38K, A40T, R67K, V68A) were identified for heavy chain of 70E11 and 9 back-mutations (R46K, D71S, Q43A, A44S, F72Y, L21M, S22T, I59V, D61A) were identified for light chain; 7 potential back-mutations (M481, M70L, R72A, R87T, R38K, A40R, Q43H) were identified for heavy chain of 149G11 and 4 back-mutations (Y42F, V89L, I21V, P49S) were identified for light chain.

As shown in Table 6, 5 humanized heavy chain variable regions and 4 humanized light chain variable regions were designed for 70E11. A total of 13 humanized antibodies were obtained as listed in Table 1 and Table 10.

TABLE 6
Back mutation design for 70E11

| | | | |
|---|---|---|---|
| VH | VH back mutation | VL | VL back mutation |
| VH1 | No back-mutation | VL1 | No back-mutation |
| VH2 | M70L, R72A | VL2 | R46K, D71S |
| VH3 | M70L, R72A, | VL3 | R46K, D71S, Q43A, |
| | M48I, T74K | | A44S, F72Y |
| VH4 | M70L, R72A, | VL4 | R46K, D71S, Q43A, |
| | M48I, T74K, | | A44S, F72Y, |
| | R38K, A40T | | L21M, S22T, 159V, |
| VH5 | M70L, R72A, | | D61A |
| | M48I, T74K, | | |
| | R38K, A40T, | | |
| | R67K, V68A | | |

As shown in Table 7, 4 humanized heavy chain variable regions and 3 humanized light chain variable regions were designed for 149G11, and a total of 7 humanized antibodies were obtained as listed in Table 1 and Table 10.

TABLE 7
Back mutation design for 149GH

| | | | |
|---|---|---|---|
| VH | VH back mutation | VL | VL back mutation |
| VH1 | No back-mutation | VL1 | No back-mutation |
| VH2 | M48I, M70L, R72A, R87T | VL2 | Y42F, V89L |
| VH3 | M48I, M70L, R72A, R87T, R38K | VL3 | Y42F, V89L, 12IV, P49S |
| VH4 | M48I, M70L, R72A, R87T, | | |
| | R38K, A40R, Q43H | | |

The sequences encoding the heavy chain variable region plus human IgG1 constant region, and the sequences encoding the light chain variable region plus human kappa constant region (amino acid sequences of heavy chain constant region and light chain constant region set forth in SEQ ID NOs: 33 and 34, respectively) were chemically synthesized and then subcloned into GS expression vectors (Invitrogen, US) using the EcoR I/Xho I and Cla I/Hind III restriction sites respectively. All expression constructs were confirmed by DNA sequencing. The EXPiCHO expression systems (Invitrogen, US) were transfected with heavy chain and light chain expressing vectors and transiently expressed 20 humanized anti-TIGIT antibodies, following the protocol described in Example 9. The humanized antibodies were purified as described in Example 3.

### Example 14 Binding Capabilities/Affinities of Humanized Anti-TIGIT Antibodies

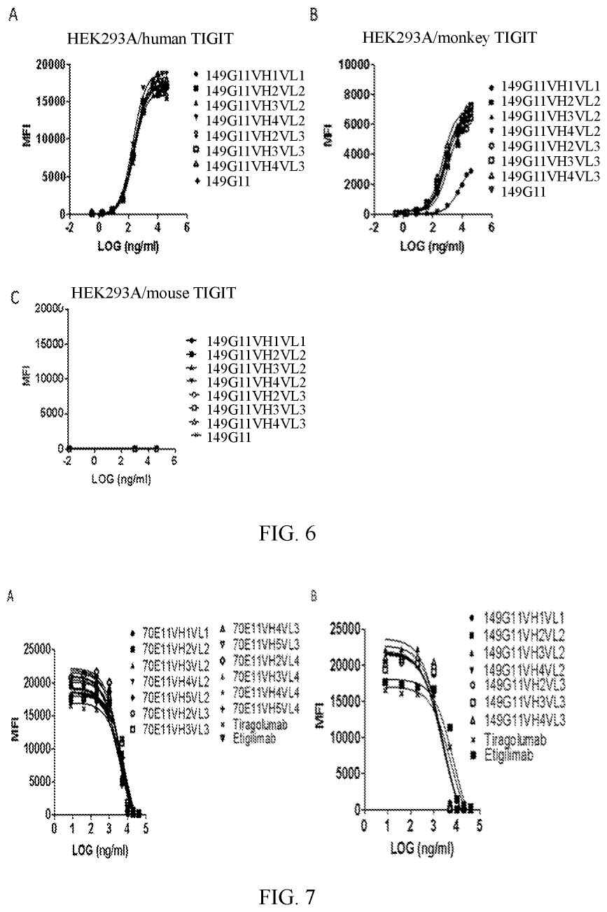

The humanized anti-TIGIT antibodies were characterized for their abilities of binding to HEK293A/human TIGIT cells, HEK293A/monkey TIGIT cells and HEK293A/mouse TIGIT cells, following the protocols described in Example 5. The results were shown in FIG. 5 and FIG. 6.

They were also tested in SPR assays for their binding affinities to human and monkey TIGITs using the BIAcore™ 8K instrument (GE Life Sciences, US). Briefly, 100-200 response units (RU) of human TIGIT (ECD)-his protein (Cat #: 10917-H08H, Sino Biological, CN) or monkey TIGIT (ECD)-hFc protein (Cat #: TIT-05254, Sino Biological, CN) were coupled to CM5 biosensor chips (Cat #: BR-1005-30, GE Life Sciences, US), followed by blocking un-reacted groups with 1M ethanolamine Serially diluted antibodies at concentrations ranging from 0.3 μM to 10 μM were injected into the SPR running buffer (HBS-EP buffer, pH7.4, Cat #: BR-1006-69, GE Life Sciences, US) at 30 μL/minute. The binding affinities were calculated with the RUs of blank controls subtracted. The association rate (kₐ) and dissociation rate (k_{d}) were calculated using the one-to-one Langmuir binding model (BIA Evaluation Software, GE Life Sciences, US). The equilibrium dissociation constant K_{D} was calculated as the k_{d}/kₐ ratio.

TABLE 8
Binding affinities of humanized anti-TIGIT antibodies to human/monkey TIGIT

| | Human TIGIT | | | Monkey TIGIT | | |
|---|---|---|---|---|---|---|
| Antibody | Kₐ | K_{d} | K_{D} | Kₐ | K_{d} | K_{D} |
| 70E11VH2VL4 | 1.3e+05 | 5.58e−05 | 4.3e−10 | 2.33e+05 | 3.35e−06 | 1.44e−11 |
| 70E11VH3VL3 | 1.56e+05 | 1.93e−05 | 1.24e−10 | 5.28e+05 | 6.97e−05 | 1.32e−10 |
| 70E11VH3VL4 | 2.40e+05 | 1.20e−05 | 5.02e−11 | 3.98e+05 | 4.13e−05 | 1.04e−10 |
| 70E11VH5VL3 | 1.89e+05 | 4.73e−05 | 2.5e−10 | 4.46e+05 | 5.92e−05 | 1.33e−10 |
| 149G11VH2VL3 | 9.71e+05 | 3.20e−06 | 3.29e−12 | 2.37e+06 | 3.37e−03 | 1.42e−09 |
| 149G11VH2VL4 | 9.47e+05 | 5.73e−06 | 6.05e−12 | 2.42e+06 | 3.40e−03 | 1.40e−09 |
| 149G11VH4VL2 | 1.01e+06 | 3.11e−06 | 3.09e−12 | 2.25e+06 | 3.42e−03 | 1.52e−09 |
| 149G11VH4VL3 | 9.76e+05 | 2.15e−06 | 2.20e−12 | 1.85e+06 | 2.56e−03 | 1.38e−09 |
| Tiragolumab | 7.43e+05 | 1.11e−06 | 1.49e−12 | 4.45e+05 | 2.77e−05 | 6.22e−11 |

As shown in FIG. 5 and FIG. 6, all the humanized anti-TIGIT antibodies had similar binding capabilities to their respective chimeric antibodies, i.e., high binding capabilities to both human and monkey TIGITs, but none of them bound mouse TIGIT.

The binding affinities of those humanized antibodies as measured by BIAcore™ were shown in Table 8.

### Example 15 Humanized Anti-TIGIT Antibodies Blocked TIGIT-PVR Interaction

The humanized anti-TIGIT antibodies were further characterized for their abilities of blocking PVR-TIGIT interaction, using the A549/human PVR cells as generated in Example 1, according to the protocol of Example 7.

The test results were shown in FIG. 7. It can be seen that all of the humanized antibodies evidently disrupted PVR-TIGIT interaction.

### Example 16 Humanized Anti-TIGIT Antibodies Activated T Cells

These humanized antibodies were further tested for their effects on T cell activation, following the protocol of Example 8. IFN-γ levels were determined by a commercially available ELISA kit (Cat #: STA00C, R&D, US) following the manufacturer's manual.

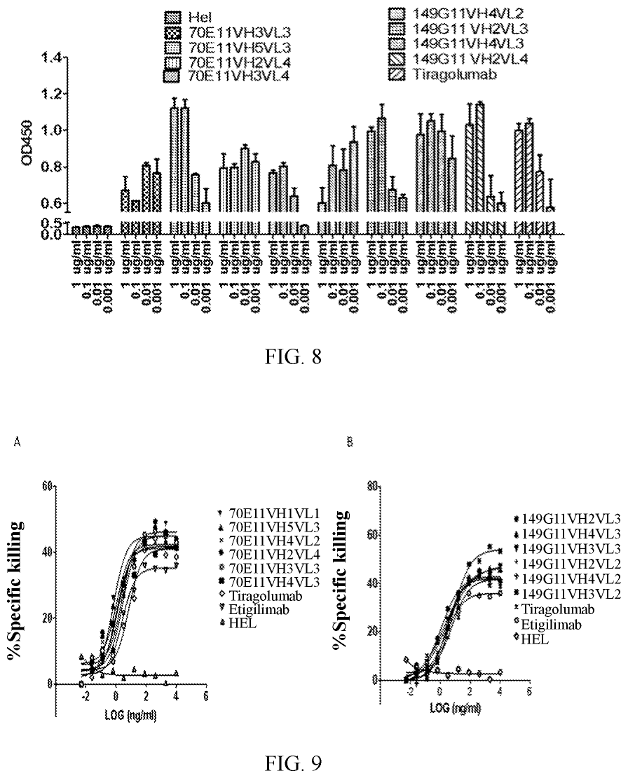

As shown in FIG. 8, all the humanized antibodies increased T cell activities, resulting in increased IFN-γ secretion. Among the antibodies as tested, 70E11VH2VL4 and 149G11VH4VL3 showed the best effects on T cell activation. Especially, the effects of these two antibodies were better than Tiragolumab at low concentrations.

### Example 17 Humanized Anti-TIGIT Antibodies Induced ADCC Against TIGIT⁺ Cells

The humanized anti-TIGIT antibodies were characterized for their capabilities to induce ADCC against HEK293A/human TIGIT cells, with NK92 cells as the effector cells, following the protocol described in Example 11.

Humanized anti-TIGIT antibodies were further tested for their capabilities to induce ADCC against HEK293A/human TIGIT cells (generated in Example 1) mediated by human PBMCs, wherein the HEK293A/human TIGIT cells were generated by transfection of GFP-expressing pLV-EGFP(2A)-Puro vectors. PBMCs from healthy human donors' blood samples were collected by density gradient centrifugation, and cultured in the medium (RIPM1640+10% FBS+300 IU IL-2) overnight. The assay was carried out by using LIVE/DEAD Fixable Dead Cell Stains Kit (Cat #: L34964, Thermo Fisher, US). Both the target cells and the effector cells (PBMCs) were centrifuged at 1200 rpm for 5 minutes. The cells were then suspended in the culture medium (RIPM1640 medium+1% FBS), and the cell viability was about 90% according to cell counting. The target cell density was adjusted to 4×10⁵/ml, and PBMC cell density was adjusted to 8×10⁶/ml. Then 50 μl HEK293A/human TIGIT cells and 50 μl PBMCs, at an effector-target ratio of 20:1, were added to each well. The antibodies were separately added to the cells, at final concentrations of 2000 ng/ml, 400 ng/ml, 80 ng/ml, 16 ng/ml, 3.2 ng/ml, 0.64 ng/ml, 0.128 ng/ml, 0.0256 ng/ml, 0.00512 ng/ml and 0.001024 ng/ml. The resultant mixtures were incubated at 37° C. for 6 h, washed with PBS for 3 times, and then incubated with LIVE/DEAD Fixable Dead Cell Stains at 37° C. for 30 minutes. The cells were washed for 3 times with PBS, and then subject to FACS. The cell death rate of GFP positive cells. i.e., the HEK293A/human TIGIT cells, was determined.

As shown in FIG. 9 and Table 9, all the humanized anti-TIGIT antibodies induced NK92 cells to specifically kill TIGIT⁺ cells, at lower EC₅₀s than Tiragolumab and Etigilimab.

TABLE 9
NK92-mediated ADCC against HEK293A/human TIGIT cells

| Antibody | EC₅₀ (M) | Antibody | EC₅₀ (M) |
|---|---|---|---|
| 149G11VH2VL3 | 7.95 E−12 | 70E11VH1VL1 | 4.36 E−12 |
| 149G11VH4VL3 | 1.77 E−11 | 70E11VH5VL3 | 7.14 E−12 |
| 149G11VH3VL3 | 1.40 E−11 | 70E11VH4VL2 | 9.12 E−12 |
| 149G11VH2VL2 | 1.62 E−11 | 70E11VH2VL4 | 1.72 E−11 |
| 149G11VH4VL2 | 1.84 E−11 | 70E11VH3VL3 | 1.91 E−11 |
| 149G11VH3VL2 | 1.30 E−11 | 70E11VH4VL3 | 7.286 E−12 |
| Tiragolumab | 4.65 E−11 | Etigilimab | 2.36 E−11 |
| HEL | / | | |

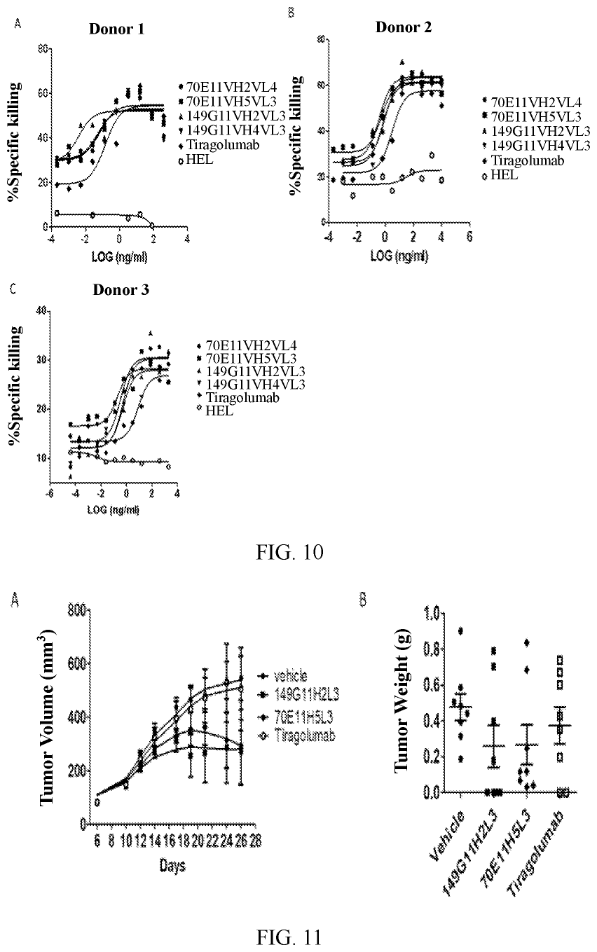

As shown in FIG. 10, all the humanized anti-TIGIT antibodies induced PBMCs to specifically kill HEK293A/human TIGIT cells, at slightly better effects than Tiragolumab.

### Example 18 Humanized Anti-TIGIT Antibodies had In Vivo Anti-Tumor Effects

The in vivo anti-tumor activities of anti-TIGIT antibodies 70E11VH5VL3 and 149G11VH2VL3 having human IgG1/kappa constant regions were studied in an animal model established by grafting HEPA1-6 murine hepatocellular carcinoma in transgenic mice with human TIGIT (GemPharmatech Co. Ltd, CN). Mice were subcutaneously injected with 7×10⁶ HEPA1-6 cells at one flank and randomly allocated into 6 groups, 8 mice per group, on Day 0. These animals were then intraperitoneally administered with 70E11VH5VL3, 149G11VH2VL3, Tiragolumab or PBS at the dose of 10 mg/kg (body weight) at Day 0, 4, 7, 11, 14 and 18.

Tumor sizes and mouse weights were monitored over time. In specific, the tumor size was determined by measuring by a caliper the length (the longest diameter) and width (the diameter perpendicular to the length) of a tumor and calculating the volume as 0.5×D×d². The test was terminated before the tumor sizes in the control group reached 3.5 cm³. One-way ANOVA was used to identify tumor size differences among groups.

The results were shown in FIG. 11. All the anti-TIGIT antibodies significantly inhibited tumor growth in mice, and 149G11VH4VL3 and 70E11VH5VL3's anti-tumor effects were better than that of Tiragolumab.

### Example 19 Humanized Anti-TIGIT Antibodies Enhanced Anti-PD-L1 Antibody's Anti-Tumor Effect

The antibodies 70E11VH2VL4, 149G11VH2VL3 and Tiragolumab were further tested for their in vivo anti-tumor activities in combination with an anti-PD-L1 antibody TECENTRIQ® Atezolizumab, using CT26 cell (colon carcinoma) grafted transgenic mice with human TIGIT (GemPharmatech Co. Ltd, CN).

On Day 0, mice were subcutaneously injected with 1×10⁶ CT26 cells at one flank and randomly allocated into 8 groups, 8 mice per group. These animals were then intraperitoneally administered with 70E11VH2VL4 (at dose of 10 mg/kg), 149G11VH2VL3 (10 mg/kg), Tiragolumab (10 mg/kg), Atezolizumab (10 mg/kg), PBS, 70E11VH2VL4 (10 mg/kg)+Atezolizumab (10 mg/kg), 149G11VH2VL3 (10 mg/kg)+Atezolizumab (10 mg/kg), or Tiragolumab (10 mg/kg)+Atezolizumab (10 mg/kg) on Day 0, 4, 7, 11, 14 and 18.

Tumor sizes and mouse weights were monitored over time. In specific, the tumor size was determined by measuring by a caliper the length (the longest diameter) and the width (the diameter perpendicular to the length) of a tumor and calculating the volume as 0.5×D×d². The test was terminated before the tumor sizes in the control group reached 3.5 cm³. One-way ANOVA was used to identify tumor size differences among groups.

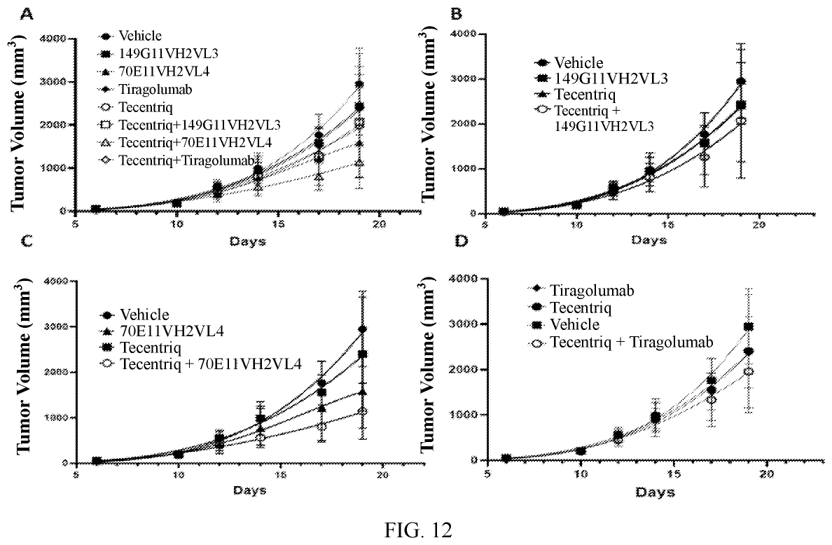

The results were shown in FIG. 12. FIG. 12(A) summarized the inhibitory effects of 70E11VH2VL4, 149G11VH2VL3, and Tiragolumab, alone or combined with Atezolizumab, on tumor growth in mice. More details of the synergistic effects of 70E11VH2VL4, 149G11VH2VL3 and Tiragolumab in combination with Atezolizumab can be found in FIGS. 12(B), 12(C) and 12(D).

As shown in FIG. 12(A), 70E11VH2VL4 and 149G11VH2VL3 significantly inhibited tumor growth in mice as compared to the vehicle group, although individual differences were found among mice in each group. FIGS. 12(B), 12(C) and 12(D) showed that the anti-tumor effects were improved when the anti-TIGIT antibodies were used in combination with an anti-PD-L1 antibody. Further, it can be seen from FIG. 12 (A) that 70E11VH2VL4 was superior to Tiragolumab in terms of the anti-tumor effect, whether used alone or in combination with an anti-PD-L1 antibody, and 149G11VH2VL3's anti-tumor effect was comparable to that of Tiragolumab, whether used alone or in combination with an anti-PD-L1 antibody.

The exemplary sequences of the disclosure are summarized below in Table 10.

TABLE 10
Sequences
Description/
Sequence/SEQ ID NO.

VH-CDR1 of mouse, chimeric and humanized 70E11 antibodies
SYNVH (SEQ ID NO: 1)
VH-CDR2 of mouse, chimeric and humanized 70E11 antibodies
TIYPGNLATSYNQKFKG (SEQ ID NO: 2)
VH-CDR3 for mouse, chimeric and humanized 70E11 antibodies
SGTMDY (SEQ ID NO: 3)
VL-CDR1 of mouse, chimeric and humanized 70E11 antibodies
RASSSISSTYLH (SEQ ID NO: 4)
VL-CDR2 of mouse, chimeric and humanized 70E11 antibodies
NTQNLAS (SEQ ID NO: 5)
VL-CDR3 of mouse, chimeric and humanized 70E11 antibodies
QQFGGYPLIT (SEQ ID NO: 6)
VH-CDR1 of mouse, chimeric and humanized 149G11 antibodies
NYWIH (SEQ ID NO: 7)
VH-CDR2 of mouse, chimeric and humanized 149G11 antibodies
DIYPGGRYSNYNEKFRG (SEQ ID NO: 8)
VH-CDR3 of mouse, chimeric and humanized 149G11 antibodies
YYESAMDF (SEQ ID NO: 9)
VL-CDRI of mouse, chimeric and humanized 149G11 antibodies
KSSQNLLYNSNQKSYLA (SEQ ID NO: 10)
VL-CDR2 of mouse, chimeric and humanized 149G11 antibodies
WASTRES (SEQ ID NO: 11)
VL-CDR3 of mouse, chimeric and humanized 149G11 antibodies
QQYYNYPFT (SEQ ID NO: 12)
VH of mouse and chimeric 70E11 antibodies
QVQLQQPGTDLVKPGASVRMSCKASGYTFTSYNVHWVKQTPGQGLEWIGTIYPGNLATSYNQKFKG
KATLTADKSSTTAYMQLSSLTSEDSAVYYCARSGTMDYWGQGTTLTVSS (SEQ ID NO: 13)
VH of humanized antibody 70E11-VH1VL1
QVQLVQSGAEVKKPGASVKVSCKASGYTFTSYNVHWVRQAPGQGLEWMGTIYPGNLATSYNQKFK
GRVTMTRDTSTSTVYMELSSLRSEDTAVYYCARSGTMDYWGQGTTVTVSS (SEQ ID NO: 14)
VH of humanized antibodies 70E11-VH2VL2, 70E11-VH2VL3, and 70E11-VH2VL4
QVQLVQSGAEVKKPGASVKVSCKASGYTFTSYNVHWVRQAPGQGLEWMGTIYPGNLATSYNQKFK
GRVTLTADTSTSTVYMELSSLRSEDTAVYYCARSGTMDYWGQGTTVTVSS (SEQ ID NO: 15)
VH of humanized antibodies 70E11-VH3VL2, 70E11-VH3VL3, and 70E11-VH3VL4
QVQLVQSGAEVKKPGASVKVSCKASGYTFTSYNVHWVRQAPGQGLEWIGTIYPGNLATSYNQKFKG
RVTLTADKSTSTVYMELSSLRSEDTAVYYCARSGTMDYWGQGTTVTVSS (SEQ ID NO: 16)
VH of humanized antibodies 70E11-VH4VL2, 70E11-VH4VL3, and 70E11-VH4VL4
QVQLVQSGAEVKKPGASVKVSCKASGYTFTSYNVHWVKQTPGQGLEWIGTIYPGNLATSYNQKFKG
RVTLTADKSTSTVYMELSSLRSEDTAVYYCARSGTMDYWGQGTTVTVSS (SEQ ID NO: 17)
VH of humanized antibodies 70E11-VH5VL2, 70E11-VH5VL3, and 70E11-VH5VL4
QVQLVQSGAEVKKPGASVKVSCKASGYTFTSYNVHWVKQTPGQGLEWIGTIYPGNLATSYNQKFKG
KATLTADKSTSTVYMELSSLRSEDTAVYYCARSGTMDYWGQGTTVTVSS (SEQ ID NO: 18)
VH of mouse and chimeric 149G11 antibodies
QVQLQQSGTELVRPGTSVKMSCRTAGYTFTNYWIHWVKQRPGHGLEWIGDIYPGGRYSNYNEKFRG
KATLTADTSSSTAYMELSSLTSEDSAIYYCARYYESAMDFWGQGTSVTVSS (SEQ ID NO: 19)
VH of humanized antibody 149G11-VH1VL1
QVQLVQSGAEVKKPGASVKVSCKASGYTFTNYWIHWVRQAPGQGLEWMGDIYPGGRYSNYNEKFR
GRVTMTRDTSTSTVYMELSSLRSEDTAVYYCARYYESAMDFWGQGTTVTVSS (SEQ ID NO: 20)
VH of humanized antibodies 149G11-VH2VL2 and 149G11-VH2VL3
QVQLVQSGAEVKKPGASVKVSCKASGYTFTNYWIHWVRQAPGQGLEWIGDIYPGGRYSNYNEKFRG
RVTLTADTSTSTVYMELSSLTSEDTAVYYCARYYESAMDFWGQGTTVTVSS (SEQ ID NO: 21)
VH of humanized antibodies 149G11-VH3VL2 and 149G11-VH3VL3
QVQLVQSGAEVKKPGASVKVSCKASGYTFTNYWIHWVKQAPGQGLEWIGDIYPGGRYSNYNEKFRG
RVTLTADTSTSTVYMELSSLTSEDTAVYYCARYYESAMDFWGQGTTVTVSS (SEQ ID NO: 22)
VH of humanized antibodies 149G11-VH4VL2 and 149G11-VH4VL3
QVQLVQSGAEVKKPGASVKVSCKASGYTFTNYWIHWVKQRPGHGLEWIGDIYPGGRYSNYNEKFRG
RVTLTADTSTSTVYMELSSLTSEDTAVYYCARYYESAMDFWGQGTTVTVSS (SEQ ID NO: 23)
VL of mouse and chimeric 70E11 antibodies
ENVVTQSPAIMSASPGEKVTMTCRASSSISSTYLHWYQQKSGASPKLWIYNTQNLASGVPARISGSGS
GTSYSLTISSVEAEDAATYYCQQFGGYPLITFGAGTKLELK (SEQ ID NO: 24)
VL of humanized antibody 70E11-VH1VL1
EIVLTQSPGTLSLSPGERATLSCRASSSISSTYLHWYQQKPGQAPRLLIYNTQNLASGIPDRFSGSGSGT
DFTLTISRLEPEDFAVYYCQQFGGYPLITFGAGTKLELK (SEQ ID NO: 25)
VL of humanized antibodies 70E11-VH2VL2, 70E11-VH3VL2, 70E11-VH4VL2 and 70E11-VH5VL2
EIVLTQSPGTLSLSPGERATLSCRASSSISSTYLHWYQQKPGQAPKLLIYNTQNLASGIPDRFSGSGSGT
SFTLTISRLEPEDFAVYYCQQFGGYPLITFGAGTKLELK (SEQ ID NO: 26)
VL of humanized antibodies 70E11-VH2VL3, 70E11-VH3VL3, 70E11-VH4VL3 and 70E11-VH5VL3
EIVLTQSPGTLSLSPGERATLSCRASSSISSTYLHWYQQKPGASPKLLIYNTQNLASGIPDRFSGSGSGTS
YTLTISRLEPEDFAVYYCQQFGGYPLITFGAGTKLELK (SEQ ID NO: 27)
VL of humanized antibodies 70E11-VH2VL4, 70E11-VH3VL4, 70E11-VH4VL4 and 70E11-VH5VL4
EIVLTQSPGTLSLSPGERATMTCRASSSISSTYLHWYQQKPGASPKLLIYNTQNLASGVPARFSGSGSG
TSYTLTISRLEPEDFAVYYCQQFGGYPLITFGAGTKLELK (SEQ ID NO: 28)
VL of mouse and chimeric 149G11 antibodies
DIVMSQSPSSLAVSVGEKVTVNCKSSQNLLYNSNQKSYLAWFQQKPGQSPKLLIYWASTRESGVPDR
FTGSGSGTDFTLTISSVKAEDLAVYYCQQYYNYPFTFGSGTKLEIK (SEQ ID NO: 29)
VL of humanized antibody 149G11-VH1VL1
DIVMTQSPDSLAVSLGERATINCKSSQNLLYNSNQKSYLAWYQQKPGQPPKLLIYWASTRESGVPDRF
SGSGSGTDFTLTISSLQAEDVAVYYCQQYYNYPFTFGAGTKLELK (SEQ ID NO: 30)
VL of humanized antibodies 149G11-VH2VL2, 149G11-VH3VL2 and 149G11-VH4VL2
DIVMTQSPDSLAVSLGERATINCKSSQNLLYNSNQKSYLAWFQQKPGQPPKLLIYWASTRESGVPDRF
SGSGSGTDFTLTISSLQAEDLAVYYCQQYYNYPFTFGAGTKLELK (SEQ ID NO: 31)
VL of humanized antibodies l49G11-VH2VL3, 149G11-VH3VL3 and 149G11-VH4VL3
DIVMTQSPDSLAVSLGERATVNCKSSQNLLYNSNQKSYLAWFQQKPGQSPKLLIYWASTRESGVPDR
FSGSGSGTDFTLTISSLQAEDLAVYYCQQYYNYPFTFGAGTKLELK (SEQ ID NO: 32)
Human IgG1 constant region
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLSPGK (SEQ ID
NO: 33)
Human kappa constant region
RTVAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC (SEQ ID NO: 34)
Human TIGIT
MRWCLLLIWAQGLRQAPLASGMMTGTIETTGNISAEKGGSIILQCHLSSTTAQVTQVNWEQQDQLLA
ICNADLGWHISPSFKDRVAPGPGLGLTLQSLTVNDTGEYFCIYHTYPDGTYTGRIFLEVLESSVAEHGA
RFQIPLLGAMAATLVVICTAVIVVVALTRKKKALRIHSVEGDLRRKSAGQEEWSPSAPSPPGSCVQAE
AAPAGLCGEQRGEDCAELHDYFNVLSYRSLGNCSFFTETG (SEQ ID NO: 35)
Monkey TIGIT
MAFLVAPPMQFVYLLKTLCVFNMVFAKPGFSETVFSHRLSFTVLSAVGYFRWQKRPHLLPVSPLGRS
MRWCLFLIWAQGLRQAPLASGMMTGTIETTGNISAKKGGSVILQCHLSSTMAQVTQVNWEQHDHSL
LAIRNAELGWHIYPAFKDRVAPGPGLGLTLQSLTMNDTGEYFCTYHTYPDGTYRGRIFLEVLESSVAE
HSARFQIPLLGAMAMMLVVICIAVIVVVVLARKKKSLRIHSVESGLQRKSTGQEEQIPSAPSPPGSCVQ
AEAAPAGLCGEQQGDDCAELHDYFNVLSYRSLGSCSFFTETG (SEQ ID NO: 36)
Mouse TIGIT
MHGWLLLVWVQGLIQAAFLATGATAGTIDTKRNISAEEGGSVILQCHFSSDTAEVTQVDWKQQDQL
LAIYSVDLGWHVASVFSDRVVPGPSLGLTFQSLTMNDTGEYFCTYHTYPGGIYKGRIFLKVQESSVAQ
FQTAPLGGTMAAVLGLICLMVTGVTVLARKKSIRMHSIESGLGRTEAEPQEWNLRSLSSPGSPVQTQT
APAGPCGEQAEDDYADPQEYFNVLSYRSLESFIAVSKTG (SEQ ID NO: 37)
Human PVR
MARAMAAAWPLLLVALLVLSWPPPGTGDVVVQAPTQVPGFLGDSVTLPCYLQVPNMEVTHVSQLT
WARHGESGSMAVFHQTQGPSYSESKRLEFVAARLGAELRNASLRMFGLRVEDEGNYTCLFVTFPQGS
RSVDIWLRVLAKPQNTAEVQKVQLTGEPVPMARCVSTGGRPPAQITWHSDLGGMPNTSQVPGFLSGT
VTVTSLWILVPSSQVDGKNVTCKVEHESFEKPQLLTVNLTVYYPPEVSISGYDNNWYLGQNEATLTC
DARSNPEPTGYNWSTTMGPLPPFAVAQGAQLLIRPVDKPINTTLICNVTNALGARQAELTVQVKEGPP
SEHSGISRNAIIFLVLGILVFLILLGIGIYFYWSKCSREVLWHCHLCPSSTEHASASANGHVSYSAVSREN
SSSQDPQTEGTR (SEQ ID NO: 38)

Having thus described in detail preferred embodiments of the present disclosure, it is to be understood that the disclosure defined by the above paragraphs is not to be limited to particular details set forth in the above description as many apparent variations thereof are possible without departing from the spirit or scope of the present disclosure.

1. An isolated monoclonal antibody, or an antigen-binding portion thereof, binding to TIGIT, comprising a heavy chain variable region comprising a VH-CDR1 region, a VH-CDR2 region and a VH-CDR3 region, and a light chain variable region comprising a VL-CDR1 region, a VL-CDR2 region and a VL-CDR3 region, wherein the VH-CDR1 region, the VH-CDR2 region, the VH-CDR3 region, the VL-CDR1 region, the VL-CDR2 region and the VL-CDR3 region comprise amino acid sequences of (1) SEQ ID NOs: 1, 2, 3, 4, 5 and 6, respectively; or (2) SEQ ID NOs: 7, 8, 9, 10, 11 and 12, respectively.

2. The isolated monoclonal antibody, or the antigen-binding portion thereof, according to claim 1, wherein the heavy chain variable region comprises an amino acid sequence having at least 95% identity to any one of SEQ ID NOs: 13-23.

3. The isolated monoclonal antibody, or the antigen-binding portion thereof, according to claim 1, wherein the light chain variable region comprises an amino acid sequence having at least 95% identity to any one of SEQ ID NO: 24-32.

4. The isolated monoclonal antibody, or the antigen-binding portion thereof, according to claim 2, wherein the heavy chain variable region and the light chain variable region comprise amino acid sequences having at least 95% identity to (1) SEQ ID NOs: 13 and 24, respectively; (2) SEQ ID NOs: 14 and 25, respectively; (3) SEQ ID NOs: 15 and 26, respectively; (4) SEQ ID NOs: 15 and 27, respectively; (5) SEQ ID NOs: 15 and 28, respectively; (6) SEQ ID NOs: 16 and 26, respectively; (7) SEQ ID NOs: 16 and 27, respectively; (8) SEQ ID NOs: 16 and 28, respectively; (9) SEQ ID NOs: 17 and 26, respectively; (10) SEQ ID NOs: 17 and 27, respectively; (11) SEQ ID NOs: 17 and 28, respectively; (12) SEQ ID NOs: 18 and 26, respectively; (13) SEQ ID NOs: 18 and 27, respectively; (14) SEQ ID NOs: 18 and 28, respectively; (15) SEQ ID NOs: 19 and 29, respectively; (16) SEQ ID NOs: 20 and 30, respectively; (17) SEQ ID NOs: 21 and 31, respectively; (18) SEQ ID NOs: 21 and 32, respectively; (19) SEQ ID NOs: 22 and 31, respectively; (20) SEQ ID NOs: 22 and 32, respectively; (21) SEQ ID NOs: 23 and 31, respectively; or (22) SEQ ID NOs: 23 and 32, respectively.

5. The isolated monoclonal antibody, or the antigen-binding portion thereof, according to claim 1, comprising a heavy chain constant region, linked to the heavy chain variable region, having an amino acid sequence having at least 95% identity to SEQ ID NO: 33, and/or a light chain constant region, linked to the light chain variable region, having an amino acid sequence having at least 95% identity to SEQ ID NO: 34.

6. The isolated monoclonal antibody, or the antigen-binding portion thereof, according to claim 1, which (a) binds human TIGIT; (b) binds monkey TIGIT; (c) does not bind to mouse TIGIT; (d) blocks TIGIT-PVR interaction; (e) promotes T cell activation; (f) induces antibody dependent cell mediated cytotoxicity (ADCC) against TIGIT positive cells; (g) has in vivo anti-tumor activity; and (h) synergizes with an anti-PD-L1 antibody.

7. The isolated monoclonal antibody, or the antigen-binding portion thereof, according to claim 1, which is mouse, chimeric or humanized.

8. A nucleic acid molecule encoding the isolated monoclonal antibody, or antigen-binding portion thereof, according to claim 1.

9. An expression vector comprising the nucleic acid molecule according claim 8.

10. A host cell comprising the expression vector according to claim 9.

11. A pharmaceutical composition comprising the isolated monoclonal antibody, or antigen-binding portion thereof, according to claim 1, and a pharmaceutically acceptable carrier.

12. A method for treating a cancer in a subject in need thereof, comprising administering to the subject the pharmaceutical composition according to claim 11.

13. The method according to claim 12, wherein the cancer is selected from the group consisting of liver cancer, rectal cancer, endometrial cancer, pancreas cancer, non-small cell lung cancer, multiple myeloma, and melanoma.

14. A method for treating an infectious disease in a subject in need thereof, comprising administering to the subject the pharmaceutical composition according to claim 11.

15. The method according to claim 14, wherein the infectious disease is caused by a viral infection.
